(19) United States
(12) Patent Application Publication
(10) Pub. No.: US 2024/0293123 A1
(43) Pub. Date: Sep. 5, 2024

(54) SURGICAL INSTRUMENT BATTERY COMPRISING A PLURALITY OF CELLS

(71) Applicant: Cilag GmbH International, Zug (CH)

(72) Inventors: Bret W. Smith, Kings Mills, OH (US); David C. Yates, Morrow, OH (US); Daniel J. Abbott, Maple Valley, WA (US); Richard F. Schwemberger, Cincinnati, OH (US); Frederick E. Shelton, IV, Hillsboro, OH (US)

(21) Appl. No.: 18/663,341

(22) Filed: May 14, 2024

Related U.S. Application Data
(63) Continuation of application No. 16/230,378, filed on Dec. 21, 2018, now Pat. No. 12,016,563.
Application No. 16/230,378 is a continuation of application No. 13/961,236, filed on Aug. 7, 2013, now Pat. No. 10,188,393.
Application No. 13/961,236 is a continuation of application No. 12/884,838, filed on Sep. 17, 2010, now Pat. No. 9,289,212.

(51) Int. Cl. A61B 17/10; A61B 1/00; A61B 1/313; A61B 17/00; A61B 17/072; A61B 17/29; A61B 17/32; A61B 18/00; A61B 18/12; H01M 50/213; H02J 7/00

(52) U.S. Cl. CPC A61B 17/105; A61B 1/00027; A61B 1/3132; A61B 17/07207; A61B 18/00; H01M 50/213; H02J 7/0042; A61B 2017/00398; A61B 2017/00734; A61B 2017/2923; A61B 2017/2927; A61B 2017/320052; A61B 2018/1226

(57) ABSTRACT

Various embodiments are directed to battery packs for use with surgical instruments. The battery packs may comprise a plurality of cells and at least a portion of the plurality of cells may not be electrically connected to one another. The battery packs may comprise a switch or other mechanism for interconnecting the plurality of cells and may also comprise, or be used in conjunction with, a discharge switch or plug configured to electrically connect an anode of the battery pack to a cathode of the battery pack, for example, via a resistive element.

### CROSS-REFERENCE TO RELATED APPLICATIONS

This application is a continuation of U.S. patent application Ser. No. 16/230,378, entitled SURGICAL INSTRUMENT BATTERY COMPRISING A PLURALITY OF CELLS, filed Dec. 21, 2018, published as U.S. Pub. No. 2019/0183506 on Jun. 20, 2019, which is a continuation of U.S. patent application Ser. No. 13/961,236, entitled SURGICAL INSTRUMENT BATTERY COMPRISING A PLURALITY OF CELLS, filed Aug. 7, 2013, issued as U.S. Pat. No. 10,188,393 on Jan. 29, 2019, which is a continuation of U.S. patent application Ser. No. 12/884,838, entitled SURGICAL INSTRUMENTS AND BATTERIES FOR SURGICAL INSTRUMENTS, filed Sep. 17, 2010, issued as U.S. Pat. No. 9,289,212 on Mar. 22, 2016, the entire disclosures of which are hereby incorporated by reference herein.

### BACKGROUND

A growing number of surgical instruments are powered by one or more battery cells. Such instruments include a variety of electrically powered implements and may be used in a variety of surgical environments. For example, battery-powered surgical instruments may include motor-driven implements (cutters, graspers, staplers, etc.) and/or non-motor driven implements (e.g., RF cutter/coagulators, ultrasonic cutter/coagulators, laser cutter/coagulators, etc.). Battery-powered instruments are also used now in various different surgical environments including, for example, endoscopic environments, laparoscopic environments, open environments, etc.

Battery-powered surgical instruments often utilize primary cells, which are pre-charged and often intended for a single discharge (e.g., one use). This avoids the difficulties associated with re-sterilizing and recharging secondary, rechargeable cells. Primary cells, however, present additional challenges related to shipping, storage and disposal.

### SUMMARY

Various embodiments may be directed to a surgical instrument comprising an end effector and a handle operatively coupled to the end effector. The handle may comprise a trigger to actuate the end effector and may also define a first cavity having a first asymmetrical cross-sectional shape and a second cavity having a second asymmetrical cross-sectional shape. A first battery pack may be positioned within the first cavity and may be in electrical contact with at least one of the handle and the end effector. The first battery pack may comprise: a first casing having a cross-sectional shape corresponding to the first asymmetrical cross-sectional shape, and a first plurality of cells electrically coupled to one another and positioned within the first casing. A second battery pack may be positioned within the second cavity and may be in electrical contact with at least one of the handle and the end effector. The second battery pack may comprise: a second casing having a cross-sectional shape corresponding to the second asymmetrical cross-sectional shape, and a second plurality of cells electrically coupled to one another and positioned within the second casing.

Also, various embodiments may be directed to a surgical system comprising a battery pack. The battery pack may comprise a casing and a plurality of cells positioned within the casing. At least a portion of the plurality of cells may not be electrically connected to one another. The battery pack may also comprise a first switch having an open position and a closed position. In the closed position, the first switch may electrically interconnect the plurality of cells. The first switch may be mechanically biased to the open position. The battery pack may further comprise a discharge switch having an open position and a closed position. The discharge switch may be positioned to, when in the closed position, electrically connect an anode of the battery pack to a cathode of the battery pack. The discharge switch may be mechanically biased to the closed position, and may be held in the open position by a portion of the casing.

According to various embodiments, the battery pack may comprise a plurality of cells, where at least a portion of the plurality of cells are not electrically connected to one another. The battery pack may further comprise a casing defining an interior cavity having at least one interior cavity wall. The at least one interior cavity wall may comprise a first electrode electrically connected to an anode of the battery pack and a second electrode electrically connected to a cathode of the battery pack. The battery pack may further comprise a battery drain positioned within the interior cavity. The battery drain may comprises first and second contacts electrically connected to one another and in contact with the at least one interior cavity wall. The battery drain may be positionable at a first position within the interior cavity where the first and second contacts are not in electrical contact with the first and second electrodes and at a second position where the first contact is in electrical contact with the first electrode and the second contact is in electrical contact with the second electrode.

Additionally, various embodiments may be directed to a surgical instrument comprising an end effector, a handle operatively coupled to the end effector, and a battery pack. The handle may comprise a trigger to actuate the end effector, and may define a cavity. The battery pack may be positioned within the cavity and may be in electrical contact with at least one of the handle and the end effector. Further, the battery pack may comprise a casing; a plurality of cells; and a movable tab. The movable tab may have a first position where it electrically separates at least a portion of the plurality of cells, and a second position where it does not electrically separate the plurality of cells.

### BRIEF DESCRIPTION OF DRAWINGS

The features of the various embodiments are set forth with particularity in the appended claims. The various embodiments, however, both as to organization and methods of operation, together with advantages thereof, may best be understood by reference to the following description, taken in conjunction with the accompanying drawings as follows:

FIGS. 1 and 2 are perspective views of one embodiment of a surgical cutting and fastening instrument;

FIG. 3 is an exploded view of one embodiment of the end effector of the surgical cutting and fastening instrument of FIGS. 1 and 2.

FIGS. 4 and 5 are exploded views of one embodiment of the end effector and shaft of the surgical cutting and fastening instrument of FIGS. 1 and 2.

FIG. 6 is a side view of one embodiment the end effector of the surgical cutting and fastening instrument of FIGS. 1 and 2.

FIG. 7 is an exploded view of one embodiment of a motor-driven endocutter.

FIGS. 8 and 9 are partial perspective views of one embodiment of the handle of the endocutter of FIG. 7.

FIG. 10 is a side view of one embodiment of the handle of the endocutter of FIG. 7.

FIG. 11 is a schematic diagram of one embodiment of an electrical circuit of a surgical cutting and fastening instrument.

FIG. 12 is a side-view of a handle of one embodiment of a power-assist motorized endocutter.

FIG. 13 is a side-view of a handle of another embodiment of a power-assist motorized endocutter.

FIGS. 14 and 15 show one embodiment of a closure trigger locking mechanism.

FIG. 16 shows another embodiment of a closure trigger locking mechanism

FIGS. 17-22 show another embodiment of a closure trigger locking mechanism.

FIGS. 23A-B show one embodiment of a universal joint (“u-joint”) that may be employed at the articulation point of a surgical instrument.

FIGS. 24A-B show one embodiment of a torsion cable that may be employed at an articulation point of a surgical instrument.

FIGS. 25-31 illustrate another embodiment of a motorized, two-stroke surgical cutting and fastening instrument with power assist.

FIGS. 32-36 illustrate one embodiment of a two-stroke, motorized surgical cutting and fastening instrument with power assist.

FIGS. 37-40 illustrate one embodiment of a motorized surgical cutting and fastening instrument with a tactile position feedback system.

FIGS. 41 and 42 illustrate two states of one embodiment of a variable sensor that may be used as the run motor sensor.

FIG. 43 illustrates one embodiment of a surgical instrument comprising a pair of asymmetrical battery packs.

FIG. 44 illustrates one embodiment of a battery pack outside of the handle of the surgical instrument of FIG. 43.

FIG. 45 illustrates one embodiment of a handle of the surgical instrument of FIG. 43 illustrating cavities for receiving battery packs.

FIG. 46 illustrates one embodiment of the battery pack of FIG. 44 showing a positive electrode contact and a negative electrode contact.

FIG. 47 illustrates one embodiment of the battery pack of FIG. 44 in conjunction with a discharge plug.

FIG. 48 illustrates a schematic diagram of one embodiment of a surgical instrument and a battery pack.

FIG. 49 illustrates an alternate embodiment of the battery pack and surgical instrument shown in FIG. 48.

FIG. 50 illustrates another embodiment of the battery pack of FIG. 48.

FIGS. 51-53 illustrate one mechanical embodiment of a battery pack implementing the schematic of the battery pack shown in FIG. 48.

FIGS. 54-59 illustrate another mechanical embodiment of a battery pack implementing the schematic of the battery pack shown in FIG. 48.

FIGS. 60 and 61 illustrates one embodiment of the battery drain of FIGS. 57-58 removed from the casing.

### DESCRIPTION

Various embodiments are directed to battery powered surgical instruments and batteries comprising features for facilitating shipping, storage and disposal. For example, according to one embodiment, a battery pack may comprise a plurality of cells mechanically and electrically coupled together within a casing having an asymmetric cross-sectional shape. The number and type of cells within the casing may be selected to reduce the power of potential accidental discharges below a threshold level. A surgical instrument for use with the battery pack may comprise a handle defining a plurality of cavities. Each cavity may have an asymmetric cross-sectional shape and at least one of the cavities may have an asymmetric cross-section shape sized to receive the battery pack. An additional cavity and/or cavities may house additional battery packs. According to various embodiments, grouping multiple cells within a single casing may reduce inconveniences associated with loading each cell into the handle individually. At the same time, limiting the number of cells grouped together may reduce safety hazards during shipping, storage and disposal.

According to various embodiments, a surgical instrument may utilize one or more battery packs, each comprising a plurality of cells and at least one switch for electrically connecting the plurality of cells. The switch may have an open position, where the cells are electrically disconnected from one another, and a closed position where the cells are electrically connected to one another. The switch may transition from the open position to the closed position when the battery pack is installed in a surgical instrument. In this way, surgical instrument may utilize power associated with a multi-cell battery. At the same time, however, the battery pack may be shipped with the switch in the open position to mitigate the available energy for a short and/or arc and, thereby, mitigate safety hazards during shipping, storage and disposal. In certain embodiments, batteries and cells described herein may have discharge switches for connecting a load across the terminals of the battery or cell to discharge the battery. For example, the discharge switch may be closed prior to disposal. In this way, the battery may discharged either prior to disposal or shortly thereafter. Accordingly, battery safety hazards due to disposal may be mitigated.

Prior to describing embodiments of the cells, batteries, battery packs, and associated surgical instruments, a detailed description of an example embodiments of a battery powered surgical instrument is provided. Although the surgical instruments described herein comprise motorized implements for cutting and stapling, it will be appreciated that the battery configurations described herein may be used with any suitable type of electrical surgical instrument including, for example, cutters, claspers, staplers, RF cutter/coagulators, ultrasonic cutter/coagulators, laser cutter/coagulators, etc.

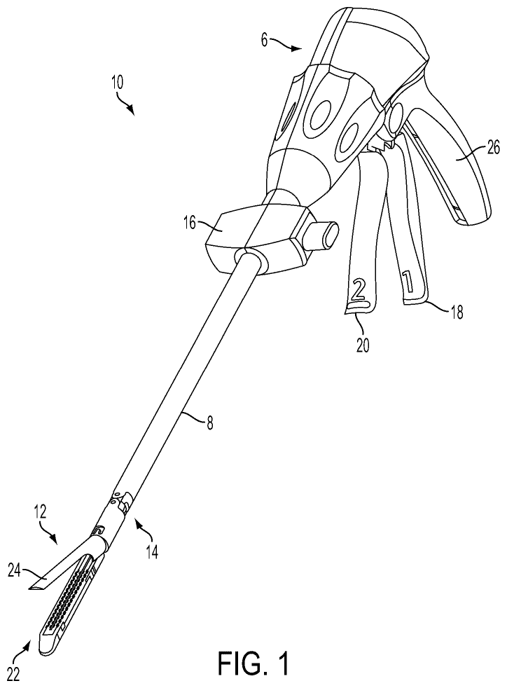

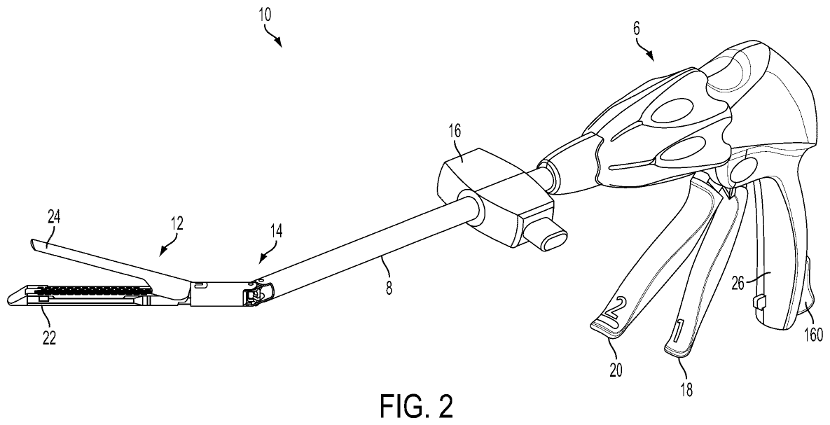

FIGS. 1 and 2 are perspective views of one embodiment of a surgical cutting and fastening instrument 10. The illustrated embodiment is an endoscopic instrument and, in general, the embodiments of the instrument 10 described herein are endoscopic surgical cutting and fastening instruments. It should be noted, however, that according to other embodiments, the instrument may be a non-endoscopic surgical cutting and fastening instrument, such as a laparoscopic or open surgical instrument.

The surgical instrument 10 depicted in FIGS. 1 and 2 comprises a handle 6, a shaft 8, and an articulating end effector 12 pivotally connected to the shaft 8 at an articulation pivot 14. An articulation control 16 may be provided adjacent to the handle 6 to effect rotation of the end effector 12 about the articulation pivot 14. In the illustrated embodiment, the end effector 12 is configured to act as an endocutter for clamping, severing and stapling tissue, although, in other embodiments, different types of end effectors may be used, such as end effectors for other types of surgical devices, such as graspers, cutters, staplers, clip appliers, access devices, drug/gene therapy devices, ultrasound, RF or laser devices, etc.

The handle 6 of the instrument 10 may include a closure trigger 18 and a firing trigger 20 for actuating the end effector 12. It will be appreciated that instruments having end effectors directed to different surgical tasks may have different numbers or types of triggers or other suitable controls for operating the end effector 12. The end effector 12 is shown separated from the handle 6 by a preferably elongate shaft 8. In one embodiment, a clinician or operator of the instrument 10 may articulate the end effector 12 relative to the shaft 8 by utilizing the articulation control 16, as described in more detail in pending U.S. patent application Ser. No. 11/329,020, filed Jan. 10, 2006, entitled SURGICAL INSTRUMENT HAVING AN ARTICULATING END EFFECTOR, by Geoffrey C. Hueil et al., now U.S. Pat. No. 7,670,334, which is incorporated herein by reference.

The end effector 12 includes in this example, among other things, a staple channel 22 and a pivotally translatable clamping member, such as an anvil 24, which are maintained at a spacing that assures effective stapling and severing of tissue clamped in the end effector 12. The handle 6 includes a pistol grip 26 towards which a closure trigger 18 is pivotally drawn by the clinician to cause clamping or closing of the anvil 24 toward the staple channel 22 of the end effector 12 to thereby clamp tissue positioned between the anvil 24 and channel 22. The firing trigger 20 is farther outboard of the closure trigger 18. Once the closure trigger 18 is locked in the closure position as further described below, the firing trigger 20 may rotate slightly toward the pistol grip 26 so that it can be reached by the operator using one hand. Then the operator may pivotally draw the firing trigger 20 toward the pistol grip 26 to cause the stapling and severing of clamped tissue in the end effector 12. In other embodiments, different types of clamping members besides the anvil 24 could be used, such as, for example, an opposing jaw, etc.

It will be appreciated that the terms “proximal” and “distal” are used herein with reference to a clinician gripping the handle 6 of an instrument 10. Thus, the end effector 12 is distal with respect to the more proximal handle 6. It will be further appreciated that, for convenience and clarity, spatial terms such as “vertical” and “horizontal” are used herein with respect to the drawings. However, surgical instruments are used in many orientations and positions, and these terms are not intended to be limiting and absolute.

The closure trigger 18 may be actuated first. Once the clinician is satisfied with the positioning of the end effector 12, the clinician may draw back the closure trigger 18 to its fully closed, locked position proximate to the pistol grip 26. The firing trigger 20 may then be actuated. The firing trigger 20 returns to the open position (shown in FIGS. 1 and 2) when the clinician removes pressure, as described more fully below. A release button 160 on the handle 6, and in this example, on the pistol grip 26 of the handle 6, when depressed may release the locked closure trigger 18.

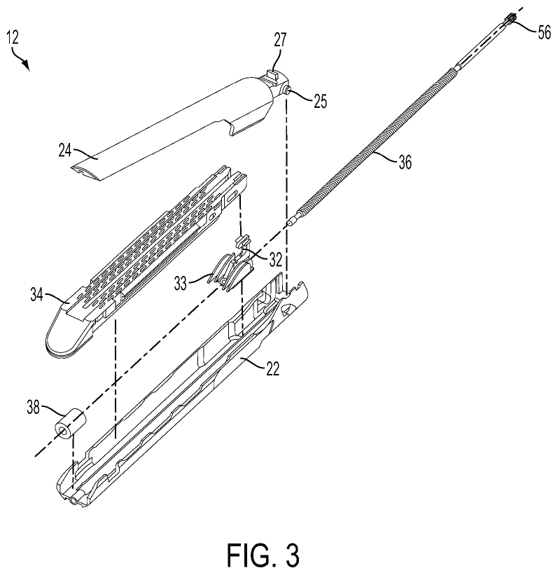

FIG. 3 is an exploded view of one embodiment of the end effector 12. As shown in the illustrated embodiment, the end effector 12 may include, in addition to the previously-mentioned channel 22 and anvil 24, a cutting instrument 32, a sled 33, a staple cartridge 34 that is removably seated in the channel 22, and a helical screw shaft 36. The cutting instrument 32 may be, for example, a knife. The anvil 24 may be pivotably opened and closed at a pivot point 25 connected to the proximate end of the channel 22. The anvil 24 may also include a tab 27 at its proximate end that is inserted into a component of the mechanical closure system (described further below) to open and close the anvil 24. When the closure trigger 18 is actuated, that is, drawn in by a user of the instrument 10, the anvil 24 may pivot about the pivot point 25 into the clamped or closed position. If clamping of the end effector 12 is satisfactory, the operator may actuate the firing trigger 20, which, as explained in more detail below, causes the knife 32 and sled 33 to travel longitudinally along the channel 22, thereby cutting tissue clamped within the end effector 12. The movement of the sled 33 along the channel 22 causes the staples of the staple cartridge 34 to be driven through the severed tissue and against the closed anvil 24, which turns the staples to fasten the severed tissue. U.S. Pat. No. 6,978,921, which issued on Dec. 27, 2005, entitled SURGICAL STAPLING INSTRUMENT INCORPORATING AN E-BEAM FIRING MECHANISM, which is incorporated herein by reference, provides more details about such two-stroke cutting and fastening instruments. According to various embodiments, the sled 33 may be an integral part of the cartridge 34, such that when the knife 32 retracts following the cutting operation, the sled 33 does not retract.

It should be noted that although the embodiments of the instrument 10 described herein employ an end effector 12 that staples the severed tissue, in other embodiments different techniques for fastening or sealing the severed tissue may be used. For example, end effectors that use RF energy or adhesives to fasten the severed tissue may also be used. U.S. Pat. No. 5,810,811, which issued Sep. 22, 1998, entitled ELECTROSURGICAL HEMOSTATIC DEVICE, which is incorporated herein by reference, discloses a cutting instrument that uses RF energy to fasten the severed tissue. U.S. patent application Ser. No. 11/267,811, entitled SURGICAL STAPLING INSTRUMENTS STRUCTURED FOR DELIVERY OF MEDICAL AGENTS, now U.S. Pat. No. 7,673,783 and U.S. patent application Ser. No. 11/267,383, entitled SURGICAL STAPLING INSTRUMENTS STRUCTURED FOR PUMP-ASSISTED DELIVERY OF MEDICAL AGENTS, now U.S. Pat. No. 7,607,557, both of which are also incorporated herein by reference, disclose cutting instruments that use adhesives to fasten the severed tissue. Accordingly, although the description herein refers to cutting/stapling operations and the like below, it should be recognized that this is an example embodiment and is not meant to be limiting. Other tissue-fastening techniques may also be used.

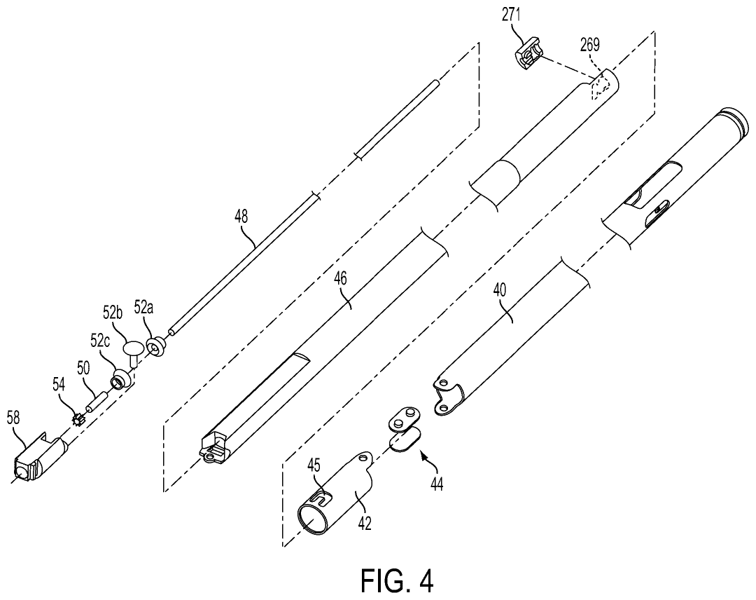

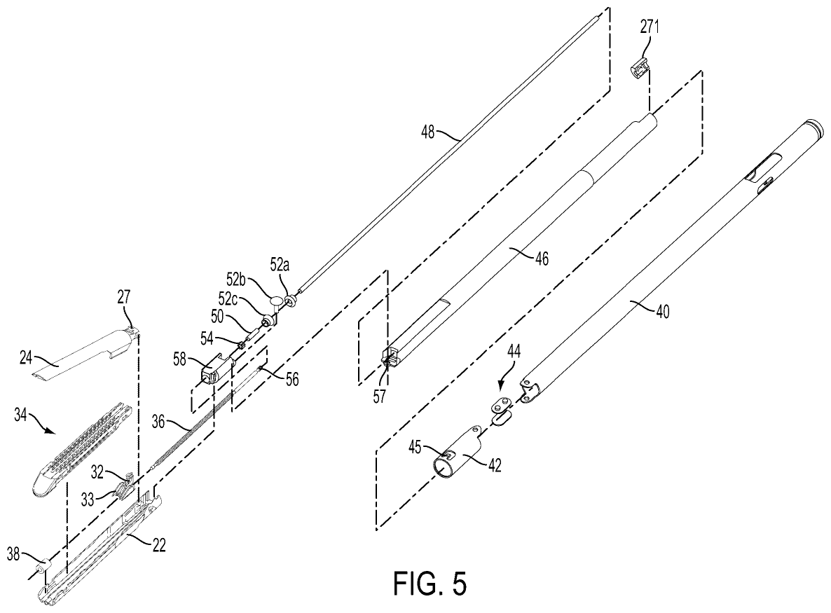

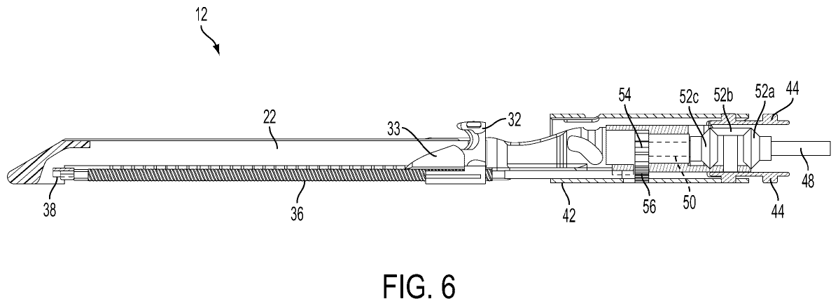

FIGS. 4 and 5 are exploded views and FIG. 6 is a side view of one embodiment of the end effector 12 and shaft 8. As shown in the illustrated embodiment, the shaft 8 may include a proximate closure tube 40 and a distal closure tube 42 pivotably linked by a pivot links 44. The distal closure tube 42 includes an opening 45 into which the tab 27 on the anvil 24 is inserted in order to open and close the anvil 24, as further described below. Disposed inside the closure tubes 40, 42 may be a proximate spine tube 46. Disposed inside the proximate spine tube 46 may be a main rotational (or proximate) drive shaft 48 that communicates with a secondary (or distal) drive shaft 50 via a bevel gear assembly 52. The secondary drive shaft 50 is connected to a drive gear 54 that engages a proximate drive gear 56 of the helical screw shaft 36. When the main drive shaft 48 is caused to rotate by actuation of the firing trigger 20 (as explained in more detail below), the bevel gear assembly 52a-c causes the secondary drive shaft 50 to rotate, which in turn, because of the engagement of the drive gears 54, 56, causes the helical screw shaft 36 to rotate, which causes the knife/sled driving member 32 to travel longitudinally along the channel 22 to cut any tissue clamped within the end effector 12. The vertical bevel gear 52b may sit and pivot in an opening 57 in the distal end of the proximate spine tube 46. A distal spine tube 58 may be used to enclose the secondary drive shaft 50 and the drive gears 54, 56. Collectively, the main drive shaft 48, the secondary drive shaft 50, and the articulation assembly (e.g., the bevel gear assembly 52a-c) are sometimes referred to herein as the “main drive shaft assembly.”

A bearing 38 is threaded on the helical drive screw 36. The bearing 36 is also connected to the knife 32. When the helical drive screw 36 forward rotates, the bearing 38 traverses the helical drive screw 36 distally, driving the cutting instrument 32 and, in the process, the sled 33 to perform the cutting/stapling operation. The sled 33 may be made of, for example, plastic, and may have a sloped distal surface. As the sled 33 traverses the channel 22, the sloped forward surface may push up or drive the staples in the staple cartridge 34 through the clamped tissue and against the anvil 24. The anvil 24 turns the staples, thereby stapling the severed tissue. When the knife 32 is retracted, the knife 32 and sled 33 may become disengaged, thereby leaving the sled 33 at the distal end of the channel 22.

Because of the lack of user feedback for the cutting/stapling operation, there is a general lack of acceptance among physicians of motor-driven surgical instruments where the cutting/stapling operation is actuated by merely pressing a button. In contrast, various embodiments may provide a motor-driven endocutter with user-feedback of the deployment, force, and/or position of the cutting instrument in the end effector.

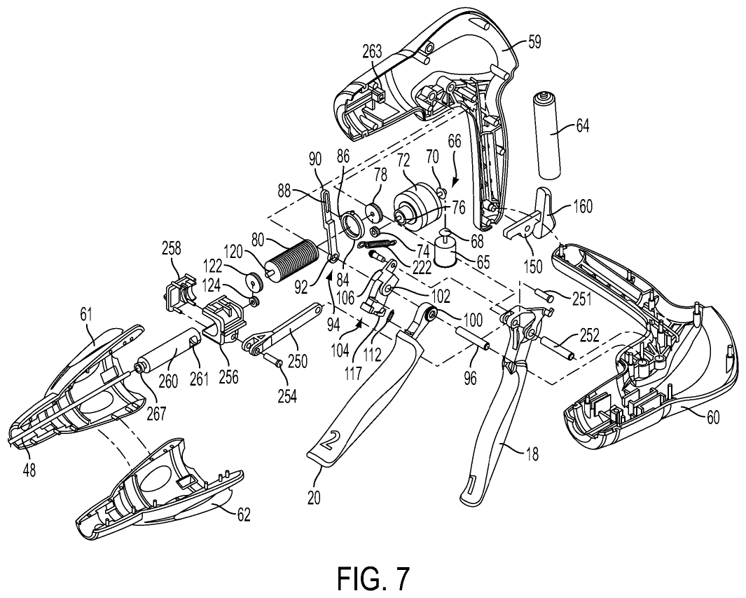

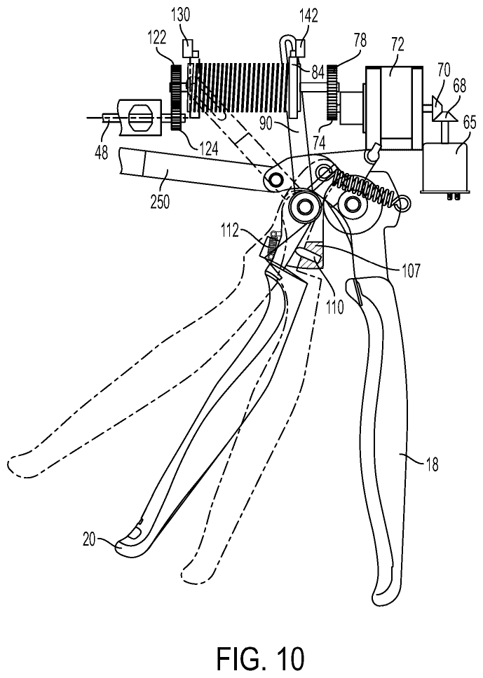

FIGS. 7-10 illustrate one embodiment of a motor-driven endocutter, and in particular the handle 6 thereof, that provides user-feedback regarding the deployment and loading force of the cutting instrument in the end effector. In addition, the embodiment may use power provided by the user in retracting the firing trigger 20 to power the device (a so-called “power assist” mode). As shown in the illustrated embodiment, the handle 6 includes exterior lower side pieces 59, 60 and exterior upper side pieces 61, 62 that fit together to form, in general, the exterior of the handle 6. A battery 64, such as a Li ion battery, may be provided in the pistol grip portion 26 of the handle 6. Although the battery 64 is illustrated as containing a single cell, it will be appreciated that the battery 64, in some embodiments, may include multiple cells connected together. The battery 64 may power a motor 65 disposed in an upper portion of the pistol grip portion 26 of the handle 6. According to various embodiments, the motor 65 may be a DC brushed driving motor having a maximum rotation of, approximately, 5000 RPM. The motor 65 may drive a 90° bevel gear assembly 66 comprising a first bevel gear 68 and a second bevel gear 70. The bevel gear assembly 66 may drive a planetary gear assembly 72. The planetary gear assembly 72 may include a pinion gear 74 connected to a drive shaft 76. The pinion gear 74 may drive a mating ring gear 78 that drives a helical gear drum 80 via a drive shaft 82. A ring 84 may be threaded on the helical gear drum 80. Thus, when the motor 65 rotates, the ring 84 is caused to travel along the helical gear drum 80 by means of the interposed bevel gear assembly 66, planetary gear assembly 72 and ring gear 78.

The handle 6 may also include a run motor sensor 110 in communication with the firing trigger 20 to detect when the firing trigger 20 has been drawn in (or “closed”) toward the pistol grip portion 26 of the handle 6 by the operator to thereby actuate the cutting/stapling operation by the end effector 12. The sensor 110 may be a proportional sensor such as, for example, a rheostat or variable resistor. When the firing trigger 20 is drawn in, the sensor 110 detects the movement, and sends an electrical signal indicative of the voltage (or power) to be supplied to the motor 65. When the sensor 110 is a variable resistor or the like, the rotation of the motor 65 may be generally proportional to the amount of movement of the firing trigger 20. That is, if the operator only draws or closes the firing trigger 20 in a little bit, the rotation of the motor 65 is relatively low. When the firing trigger 20 is fully drawn in (or in the fully closed position), the rotation of the motor 65 is at its maximum. In other words, the harder the user pulls on the firing trigger 20, the more voltage is applied to the motor 65, causing greater rates of rotation.

The handle 6 may include a middle handle piece 104 adjacent to the upper portion of the firing trigger 20. The handle 6 also may comprise a bias spring 112 connected between posts on the middle handle piece 104 and the firing trigger 20. The bias spring 112 may bias the firing trigger 20 to its fully open position. In that way, when the operator releases the firing trigger 20, the bias spring 112 will pull the firing trigger 20 to its open position, thereby removing actuation of the sensor 110, thereby stopping rotation of the motor 65. Moreover, by virtue of the bias spring 112, any time a user closes the firing trigger 20, the user will experience resistance to the closing operation, thereby providing the user with feedback as to the amount of rotation exerted by the motor 65. Further, the operator could stop retracting the firing trigger 20 to thereby remove force from the sensor 100, to thereby stop the motor 65. As such, the user may stop the deployment of the end effector 12, thereby providing a measure of control of the cutting/fastening operation to the operator.

The distal end of the helical gear drum 80 includes a distal drive shaft 120 that drives a ring gear 122, which mates with a pinion gear 124. The pinion gear 124 is connected to the main drive shaft 48 of the main drive shaft assembly. In that way, rotation of the motor 65 causes the main drive shaft assembly to rotate, which causes actuation of the end effector 12, as described above.

The ring 84 threaded on the helical gear drum 80 may include a post 86 that is disposed within a slot 88 of a slotted arm 90. The slotted arm 90 has an opening 92 its opposite end 94 that receives a pivot pin 96 that is connected between the handle exterior side pieces 59, 60. The pivot pin 96 is also disposed through an opening 100 in the firing trigger 20 and an opening 102 in the middle handle piece 104.

In addition, the handle 6 may include a reverse motor (or end-of-stroke sensor) 130 and a stop motor (or beginning-of-stroke) sensor 142. In various embodiments, the reverse motor sensor 130 may be a limit switch located at the distal end of the helical gear drum 80 such that the ring 84 threaded on the helical gear drum 80 contacts and trips the reverse motor sensor 130 when the ring 84 reaches the distal end of the helical gear drum 80. The reverse motor sensor 130, when activated, sends a signal to the motor 65 to reverse its rotation direction, thereby withdrawing the knife 32 of the end effector 12 following the cutting operation.

The stop motor sensor 142 may be, for example, a normally-closed limit switch. In various embodiments, it may be located at the proximate end of the helical gear drum 80 so that the ring 84 trips the switch 142 when the ring 84 reaches the proximate end of the helical gear drum 80.

In operation, when an operator of the instrument 10 pulls back the firing trigger 20, the sensor 110 detects the deployment of the firing trigger 20 and sends a signal to the motor 65 to cause forward rotation of the motor 65 at, for example, a rate proportional to how hard the operator pulls back the firing trigger 20. The forward rotation of the motor 65 in turn causes the ring gear 78 at the distal end of the planetary gear assembly 72 to rotate, thereby causing the helical gear drum 80 to rotate, causing the ring 84 threaded on the helical gear drum 80 to travel distally along the helical gear drum 80. The rotation of the helical gear drum 80 also drives the main drive shaft assembly as described above, which in turn causes deployment of the knife 32 in the end effector 12. That is, the knife 32 and sled 33 are caused to traverse the channel 22 longitudinally, thereby cutting tissue clamped in the end effector 12. Also, the stapling operation of the end effector 12 is caused to happen in embodiments where a stapling-type end effector is used.

By the time the cutting/stapling operation of the end effector 12 is complete, the ring 84 on the helical gear drum 80 will have reached the distal end of the helical gear drum 80, thereby causing the reverse motor sensor 130 to be tripped, which sends a signal to the motor 65 to cause the motor 65 to reverse its rotation. This in turn causes the knife 32 to retract, and also causes the ring 84 on the helical gear drum 80 to move back to the proximate end of the helical gear drum 80.

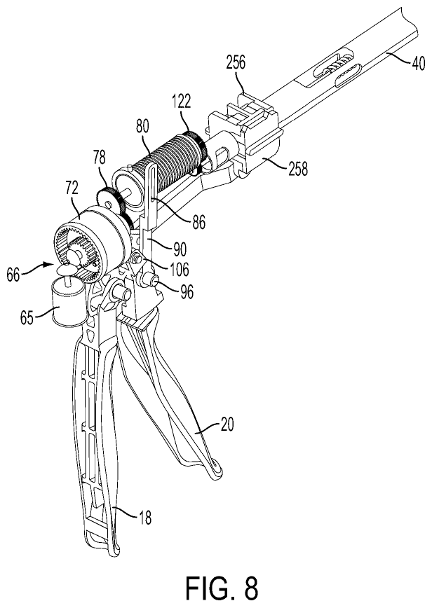

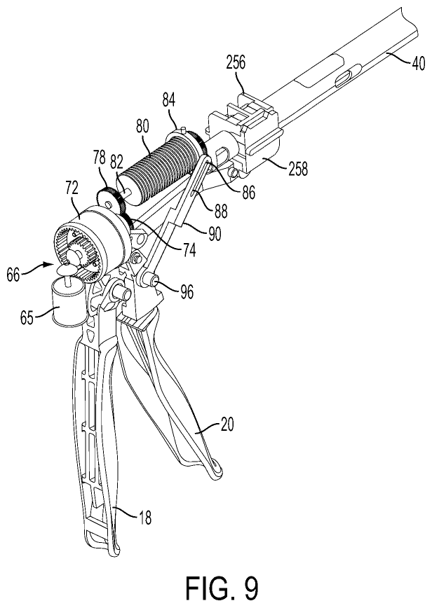

The middle handle piece 104 includes a backside shoulder 106 that engages the slotted arm 90 as best shown in FIGS. 8 and 9. The middle handle piece 104 also has a forward motion stop 107 that engages the firing trigger 20. The movement of the slotted arm 90 is controlled, as explained above, by rotation of the motor 65. When the slotted arm 90 rotates CCW as the ring 84 travels from the proximate end of the helical gear drum 80 to the distal end, the middle handle piece 104 will be free to rotate CCW. Thus, as the user draws in the firing trigger 20, the firing trigger 20 will engage the forward motion stop 107 of the middle handle piece 104, causing the middle handle piece 104 to rotate CCW. Due to the backside shoulder 106 engaging the slotted arm 90, however, the middle handle piece 104 will only be able to rotate CCW as far as the slotted arm 90 permits. In that way, if the motor 65 should stop rotating for some reason, the slotted arm 90 will stop rotating, and the user will not be able to further draw in the firing trigger 20 because the middle handle piece 104 will not be free to rotate CCW due to the slotted arm 90.

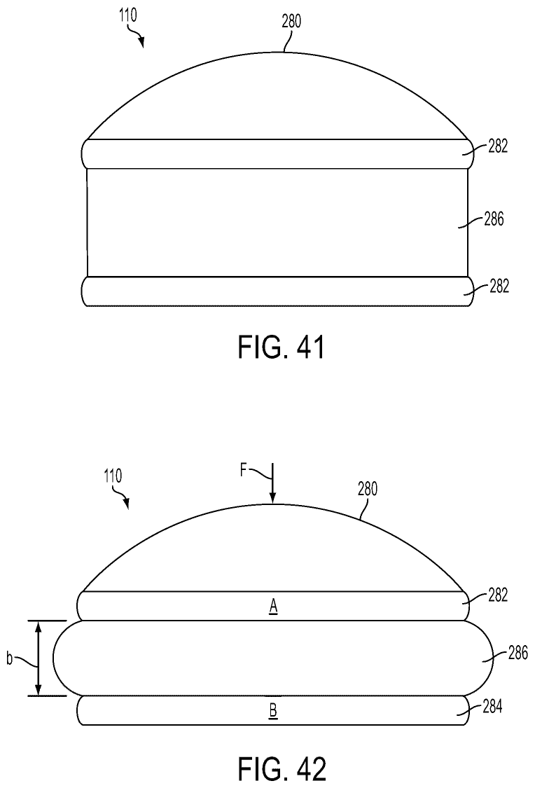

FIGS. 41 and 42 illustrate two states of one embodiment of a variable sensor that may be used as the run motor sensor 110. The sensor 110 may include a face portion 280, a first electrode (A) 282, a second electrode (B) 284, and a compressible dielectric material 286 (e.g., EAP) between the electrodes 282, 284. The sensor 110 may be positioned such that the face portion 280 contacts the firing trigger 20 when retracted. Accordingly, when the firing trigger 20 is retracted, the dielectric material 286 is compressed, as shown in FIG. 42, such that the electrodes 282, 284 are closer together. Since the distance “b” between the electrodes 282, 284 is directly related to the impedance between the electrodes 282, 284, the greater the distance the more impedance, and the closer the distance the less impedance. In that way, the amount that the dielectric material 286 is compressed due to retraction of the firing trigger 20 (denoted as force “F” in FIG. 42) is proportional to the impedance between the electrodes 282, 284, which can be used to proportionally control the motor 65.

Components of an example closure system for closing (or clamping) the anvil 24 of the end effector 12 by retracting the closure trigger 18 are also shown in FIGS. 7-10. In the illustrated embodiment, the closure system includes a yoke 250 connected to the closure trigger 18 by a pin 251 that is inserted through aligned openings in both the closure trigger 18 and the yoke 250. A pivot pin 252, about which the closure trigger 18 pivots, is inserted through another opening in the closure trigger 18 which is offset from where the pin 251 is inserted through the closure trigger 18. Thus, retraction of the closure trigger 18 causes the upper part of the closure trigger 18, to which the yoke 250 is attached via the pin 251, to rotate CCW. The distal end of the yoke 250 is connected, via a pin 254, to a first closure bracket 256. The first closure bracket 256 connects to a second closure bracket 258. Collectively, the closure brackets 256, 258 define an opening in which the proximate end of the proximate closure tube 40 (see FIG. 4) is seated and held such that longitudinal movement of the closure brackets 256, 258 causes longitudinal motion by the proximate closure tube 40. The instrument 10 also includes a closure rod 260 disposed inside the proximate closure tube 40. The closure rod 260 may include a window 261 into which a post 263 on one of the handle exterior pieces, such as exterior lower side piece 59 in the illustrated embodiment, is disposed to fixedly connect the closure rod 260 to the handle 6. In that way, the proximate closure tube 40 is capable of moving longitudinally relative to the closure rod 260. The closure rod 260 may also include a distal collar 267 that fits into a cavity 269 in proximate spine tube 46 and is retained therein by a cap 271 (see FIG. 4).

In operation, when the yoke 250 rotates due to retraction of the closure trigger 18, the closure brackets 256, 258 cause the proximate closure tube 40 to move distally (e.g., away from the handle end of the instrument 10), which causes the distal closure tube 42 to move distally, which causes the anvil 24 to rotate about the pivot point 25 into the clamped or closed position. When the closure trigger 18 is unlocked from the locked position, the proximate closure tube 40 is caused to slide proximally, which causes the distal closure tube 42 to slide proximally, which by virtue of the tab 27 being inserted in the window 45 of the distal closure tube 42, causes the anvil 24 to pivot about the pivot point 25 into the open or unclamped position. In that way, by retracting and locking the closure trigger 18, an operator may clamp tissue between the anvil 24 and channel 22, and may unclamp the tissue following the cutting/stapling operation by unlocking the closure trigger 18 from the locked position.

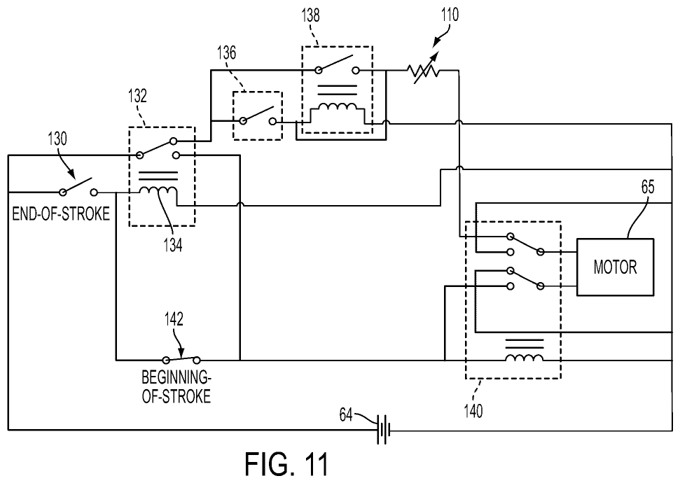

FIG. 11 is a schematic diagram of one embodiment of an electrical circuit of the instrument 10. When an operator initially pulls in the firing trigger 20 after locking the closure trigger 18, the sensor 110 is activated, allowing current to flow therethrough. If the normally-open reverse motor sensor switch 130 is open (meaning the end of the end effector stroke has not been reached), current will flow to a single pole, double throw relay 132. Since the reverse motor sensor switch 130 is not closed, the coil 134 of the relay 132 will not be energized, so the relay 132 will be in its non-energized state. The circuit also includes a cartridge lockout sensor switch 136. If the end effector 12 includes a staple cartridge 34, the sensor switch 136 will be in the closed state, allowing current to flow. Otherwise, if the end effector 12 does not include a staple cartridge 34, the sensor switch 136 will be open, thereby preventing the battery 64 from powering the motor 65.

When the staple cartridge 34 is present, the sensor switch 136 is closed, which energizes a single pole, single throw relay 138. When the relay 138 is energized, current flows through the relay 138, through the variable resistor sensor 110, and to the motor 65 via a double pole, double throw relay 140, thereby powering the motor 65 and allowing it to rotate in the forward direction.

When the end effector 12 reaches the end of its stroke, the reverse motor sensor 130 will be activated, thereby closing the switch 130 and energizing the relay 132. This causes the relay 132 to assume its energized state (not shown in FIG. 11), which causes current to bypass the cartridge lockout sensor switch 136 and variable resistor 110, and instead causes current to flow to both the normally-closed double pole, double throw relay 140 and back to the motor 65, but in a manner, via the relay 140, that causes the motor 65 to reverse its rotational direction.

Because the stop motor sensor switch 142 is normally-closed, current will flow back to the relay 132 to keep it energized until the switch 142 opens. When the knife 32 is fully retracted, the stop motor sensor switch 142 is activated, causing the switch 142 to open, thereby removing power from the motor 65.

In other embodiments, rather than a proportional-type sensor 110, an on-off type sensor could be used. In such embodiments, the rate of rotation of the motor 65 would not be proportional to the force applied by the operator. Rather, the motor 65 would generally rotate at a constant rate. But the operator would still experience force feedback because the firing trigger 20 is geared into the gear drive train.

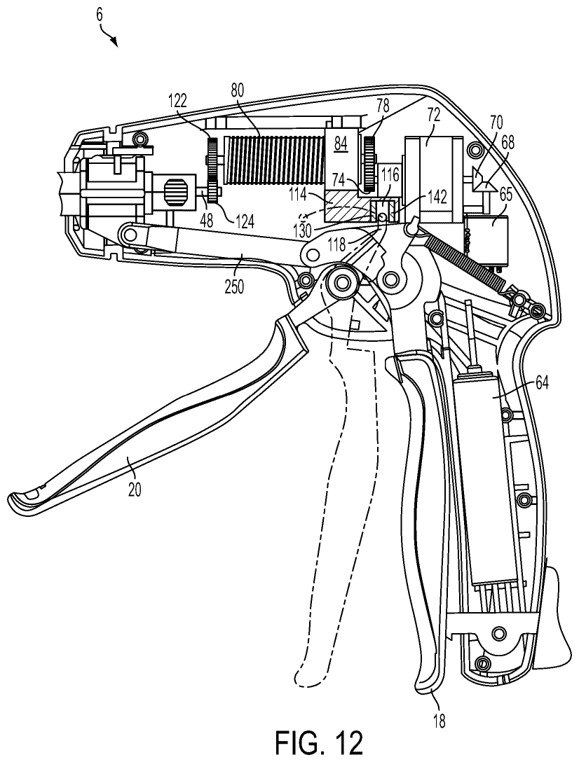

FIG. 12 is a side-view of the handle 6 of a power-assist motorized endocutter according to another embodiment. The embodiment of FIG. 12 is similar to that of FIGS. 7-10 except that in the embodiment of FIG. 12, there is no slotted arm 90 connected to the ring 84 threaded on the helical gear drum 80. Instead, in the embodiment of FIG. 12, the ring 84 includes a sensor portion 114 that moves with the ring 84 as the ring 84 advances down (and back) on the helical gear drum 80. The sensor portion 114 includes a notch 116. The reverse motor sensor 130 may be located at the distal end of the notch 116 and the stop motor sensor 142 may be located at the proximate end of the notch 116. As the ring 84 moves down the helical gear drum 80 (and back), the sensor portion 114 moves with it. Further, as shown in FIG. 12, the middle piece 104 may have an arm 118 that extends into the notch 116.

In operation, as an operator of the instrument 10 retracts in the firing trigger 20 toward the pistol grip 26, the run motor sensor 110 detects the motion and sends a signal to power the motor 65, which causes, among other things, the helical gear drum 80 to rotate. As the helical gear drum 80 rotates, the ring 84 threaded on the helical gear drum 80 advances (or retracts, depending on the rotation). Also, due to the pulling in of the firing trigger 20, the middle piece 104 is caused to rotate CCW with the firing trigger 20 due to the forward motion stop 107 that engages the firing trigger 20. The CCW rotation of the middle piece 104 cause the arm 118 to rotate CCW with the sensor portion 114 of the ring 84 such that the arm 118 stays disposed in the notch 116. When the ring 84 reaches the distal end of the helical gear drum 80, the arm 118 will contact and thereby trip the reverse motor sensor 130. Similarly, when the ring 84 reaches the proximate end of the helical gear drum 80, the arm 118 will contact and thereby trip the stop motor sensor 142. Such actions may reverse and stop the motor 65, respectively, as described above.

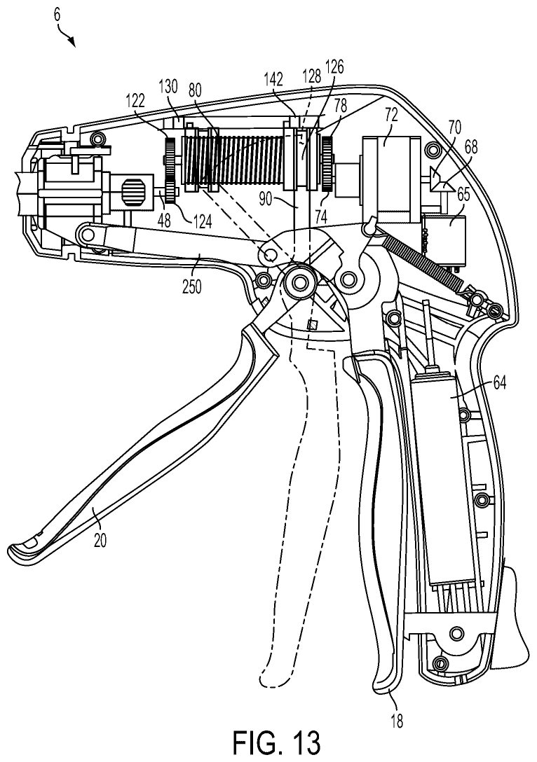

FIG. 13 is a side-view of the handle 6 of a power-assist motorized endocutter according to another embodiment. The embodiment of FIG. 13 is similar to that of FIGS. 7-10 except that in the embodiment of FIG. 13, there is no slot in the arm 90. Instead, the ring 84 threaded on the helical gear drum 80 includes a vertical channel 126. Instead of a slot, the arm 90 includes a post 128 that is disposed in the channel 126. As the helical gear drum 80 rotates, the ring 84 threaded on the helical gear drum 80 advances (or retracts, depending on the rotation). The arm 90 rotates CCW as the ring 84 advances due to the post 128 being disposed in the channel 126, as shown in FIG. 13.

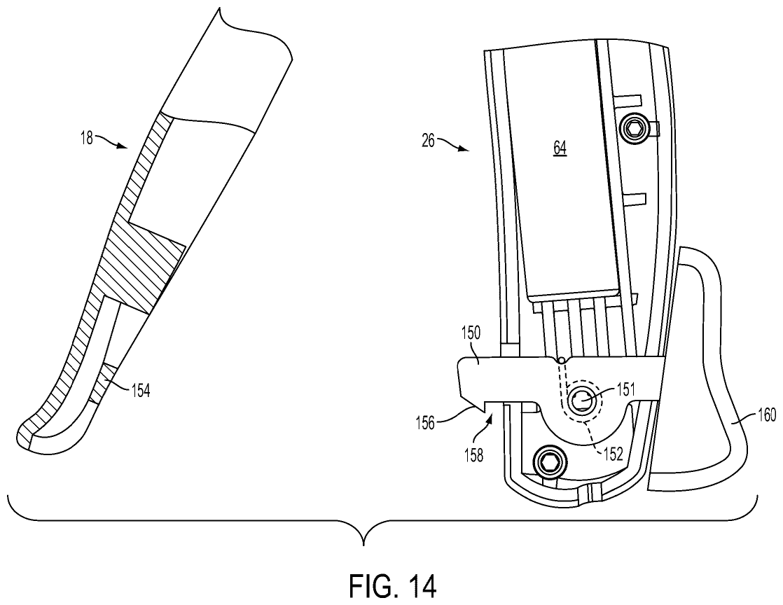

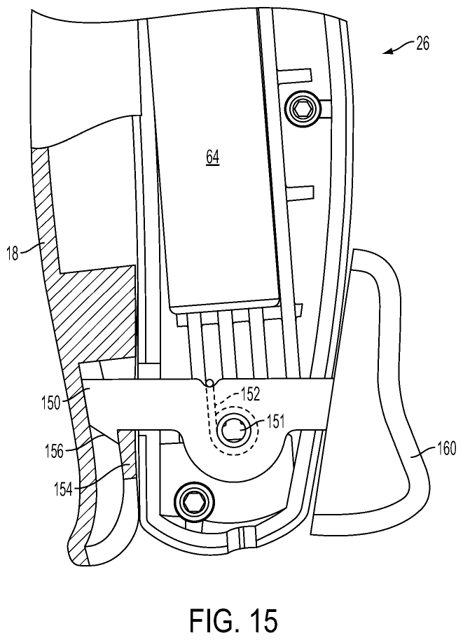

As mentioned above, in using a two-stroke motorized instrument, the operator first pulls back and locks the closure trigger 18. FIGS. 14 and 15 show one embodiment of a closure trigger 18 locking mechanism for locking the closure trigger 18 to the pistol grip portion 26 of the handle 6. In the illustrated embodiment, the pistol grip portion 26 includes a hook 150 that is biased to rotate CCW about a pivot point 151 by a torsion spring 152. Also, the closure trigger 18 includes a closure bar 154. As the operator draws in the closure trigger 18, the closure bar 154 engages a sloped portion 156 of the hook 150, thereby rotating the hook 150 upward (or CW in FIGS. 14-15) until the closure bar 154 completely passes the sloped portion 156 into a recessed notch 158 of the hook 150, which locks the closure trigger 18 in place. The operator may release the closure trigger 18 by pushing down on a slide button release 160 on the back or opposite side of the pistol grip portion 26. Pushing down the slide button release 160 rotates the hook 150 CW such that the closure bar 154 is released from the recessed notch 158.

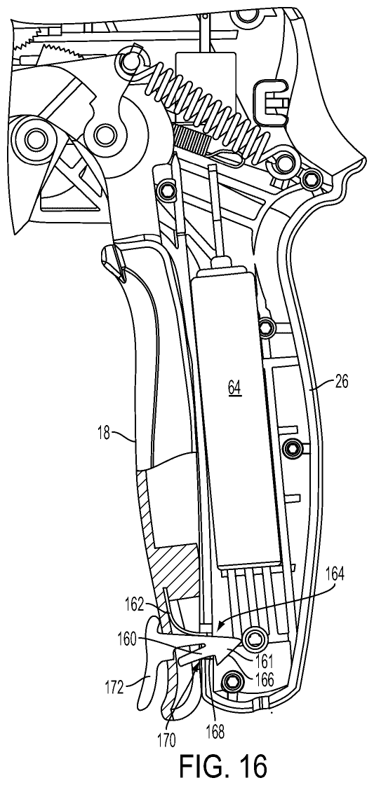

FIG. 16 shows another closure trigger locking mechanism according to various embodiments. In the embodiment of FIG. 16, the closure trigger 18 includes a wedge 160 having an arrow-head portion 161. The arrow-head portion 161 is biased downward (or CW) by a leaf spring 162. The wedge 160 and leaf spring 162 may be made from, for example, molded plastic. When the closure trigger 18 is retracted, the arrow-head portion 161 is inserted through an opening 164 in the pistol grip portion 26 of the handle 6. A lower chamfered surface 166 of the arrow-head portion 161 engages a lower sidewall 168 of the opening 164, forcing the arrow-head portion 161 to rotate CCW. Eventually the lower chamfered surface 166 fully passes the lower sidewall 168, removing the CCW force on the arrow-head portion 161, causing the lower sidewall 168 to slip into a locked position in a notch 170 behind the arrow-head portion 161.

To unlock the closure trigger 18, a user presses down on a button 172 on the opposite side of the closure trigger 18, causing the arrow-head portion 161 to rotate CCW and allowing the arrow-head portion 161 to slide out of the opening 164.

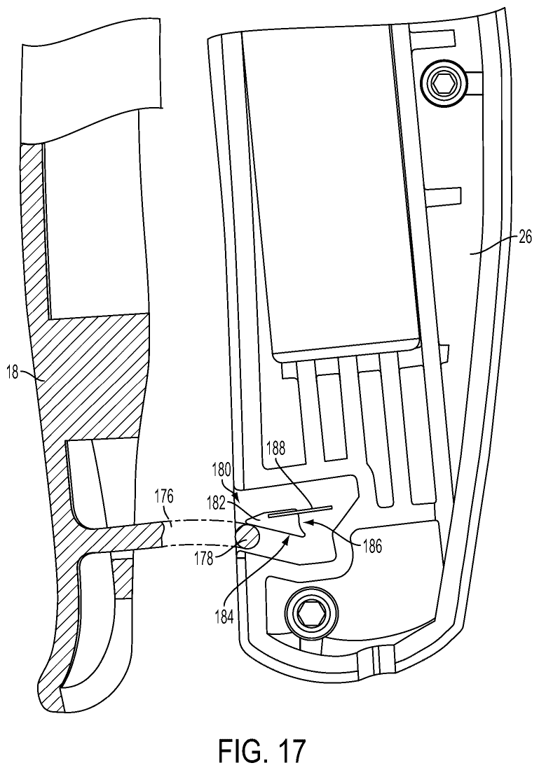

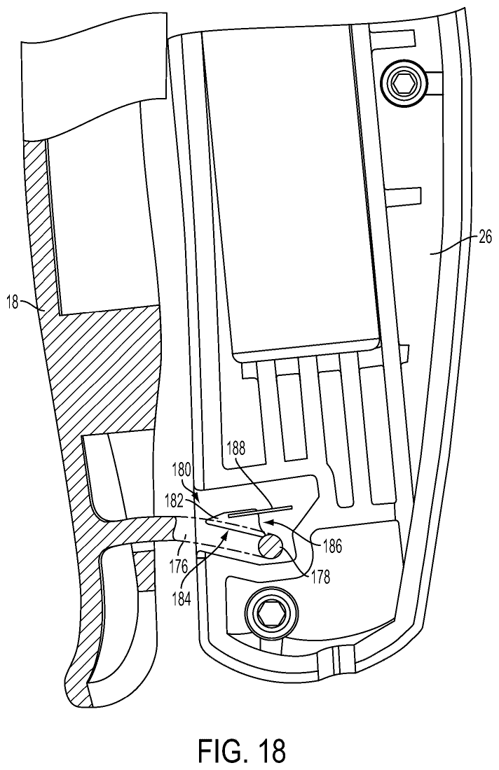

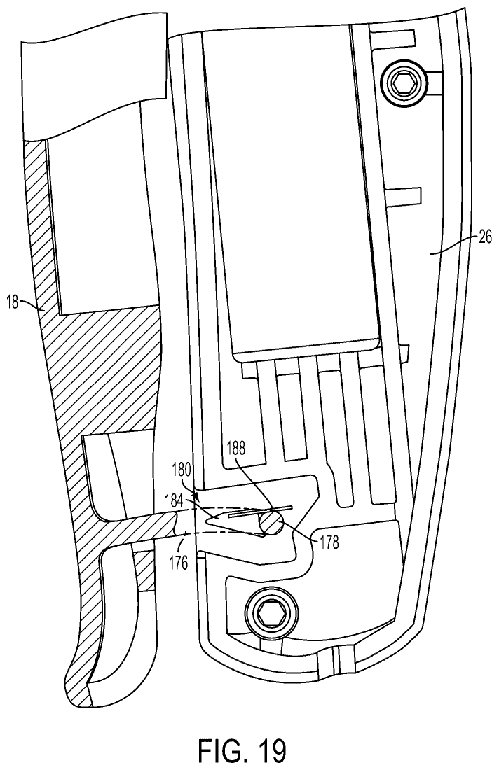

FIGS. 17-22 show another embodiment of a closure trigger locking mechanism. As shown in this embodiment, the closure trigger 18 includes a flexible longitudinal arm 176 that includes a lateral pin 178 extending therefrom. The arm 176 and pin 178 may be made from molded plastic, for example. The pistol grip portion 26 of the handle 6 includes an opening 180 with a laterally extending wedge 182 disposed therein. When the closure trigger 18 is retracted, the pin 178 engages the wedge 182, and the pin 178 is forced downward (e.g., the arm 176 is rotated CW) by the lower surface 184 of the wedge 182, as shown in FIGS. 17 and 18. When the pin 178 fully passes the lower surface 184, the CW force on the arm 176 is removed, and the pin 178 is rotated CCW such that the pin 178 comes to rest in a notch 186 behind the wedge 182, as shown in FIG. 19, thereby locking the closure trigger 18. The pin 178 is further held in place in the locked position by a flexible stop 188 extending from the wedge 184.

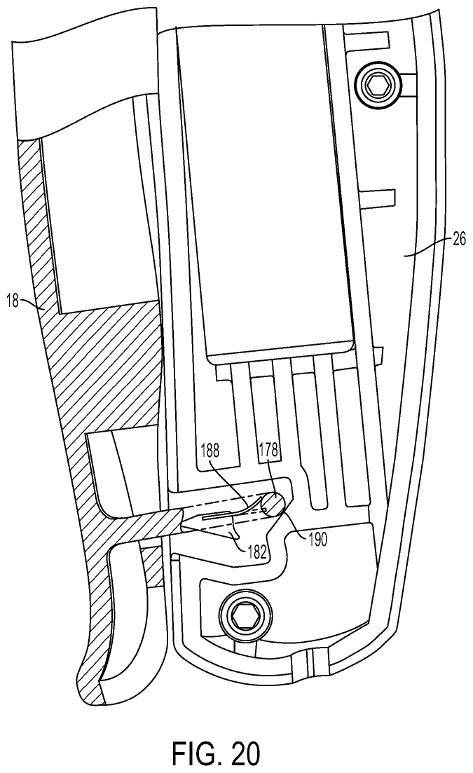

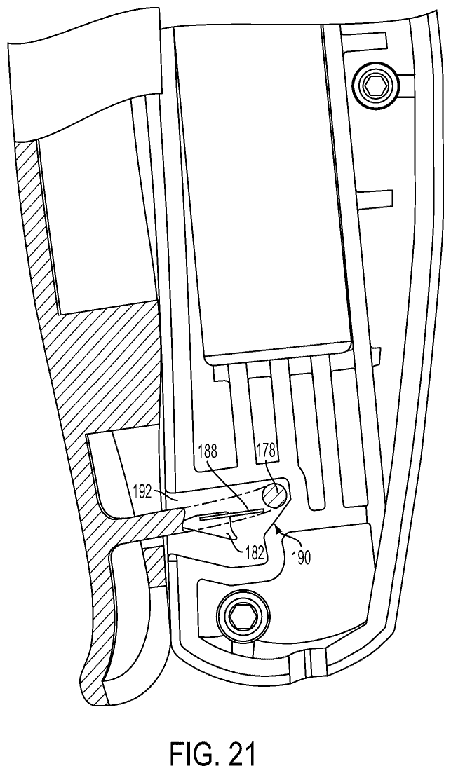

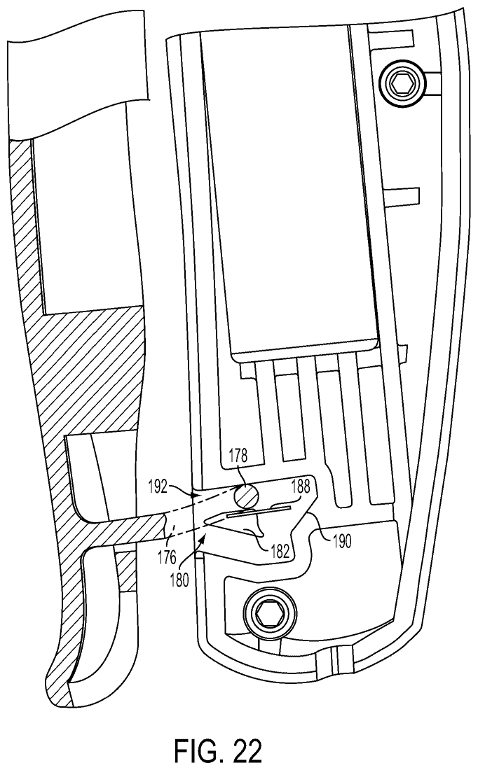

To unlock the closure trigger 18, the operator may further squeeze the closure trigger 18, causing the pin 178 to engage a sloped backwall 190 of the opening 180, forcing the pin 178 upward past the flexible stop 188, as shown in FIGS. 20 and 21. The pin 178 is then free to travel out an upper channel 192 in the opening 180 such that the closure trigger 18 is no longer locked to the pistol grip portion 26, as shown in FIG. 22.

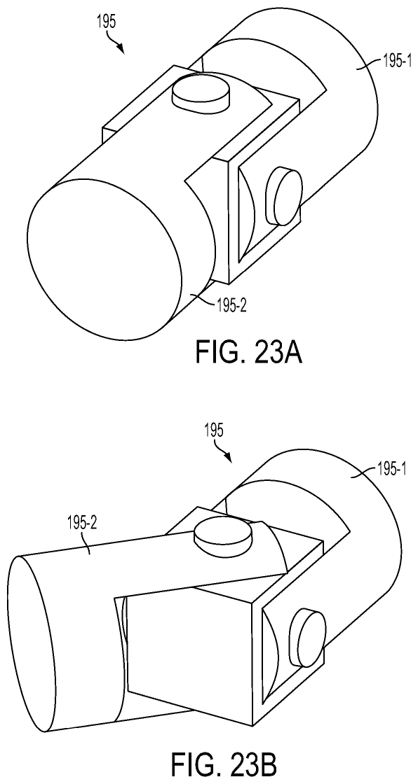

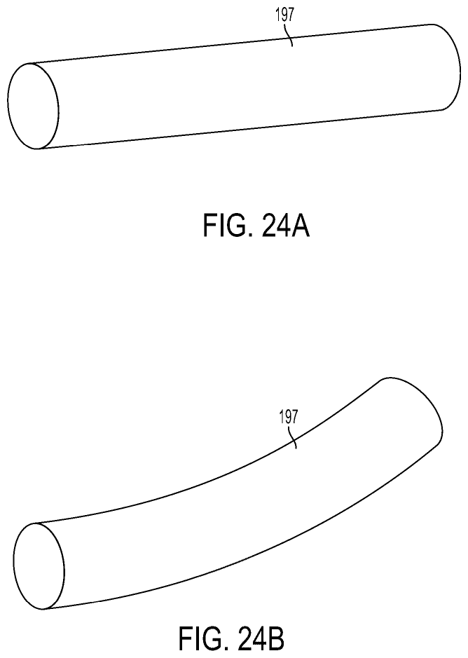

FIGS. 23A-B show a universal joint (“u-joint”) 195 that may be employed at the articulation point of a surgical instrument, such as the instrument 10. The second piece 195-2 of the u-joint 195 rotates in a horizontal plane in which the first piece 195-1 lies. FIG. 23A shows the u-joint 195 in a linear (180°) orientation and FIG. 23B shows the u-joint 195 at approximately a 150° orientation. The u-joint 195 may be used instead of the bevel gears 52a-c (see FIG. 4, for example) at the articulation point 14 of the main drive shaft assembly to articulate the end effector 12. FIGS. 24A-B show a torsion cable 197 that may be used in lieu of both the bevel gears 52a-c and the u-joint 195 to realize articulation of the end effector 12.

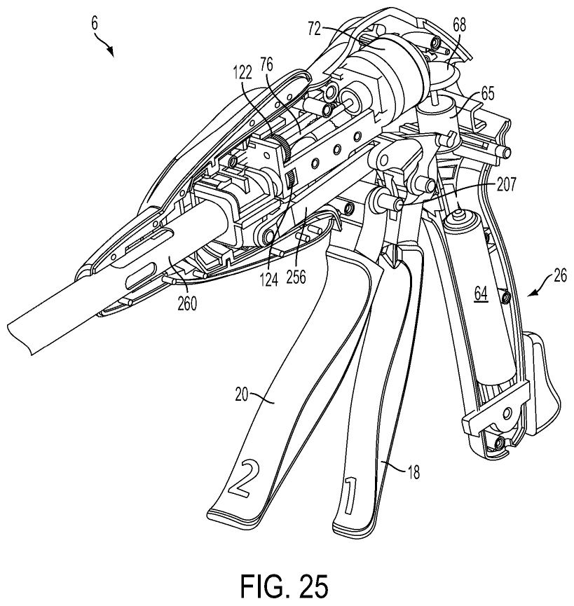

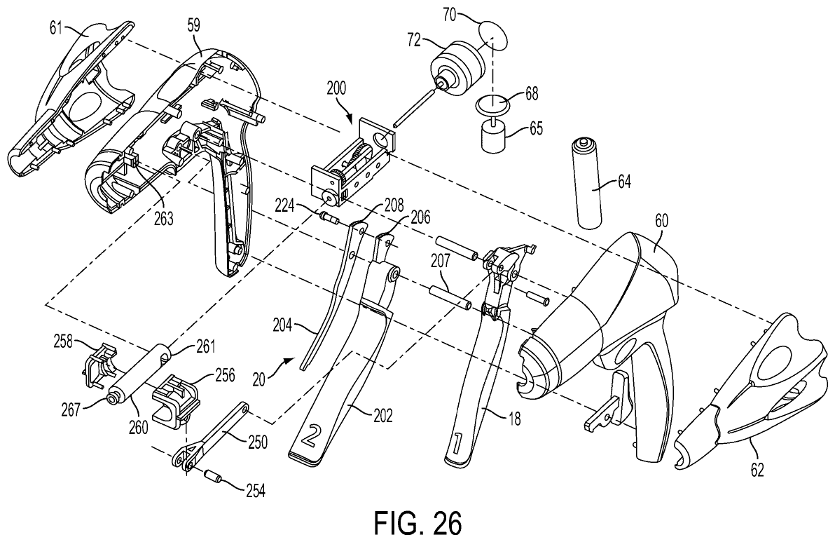

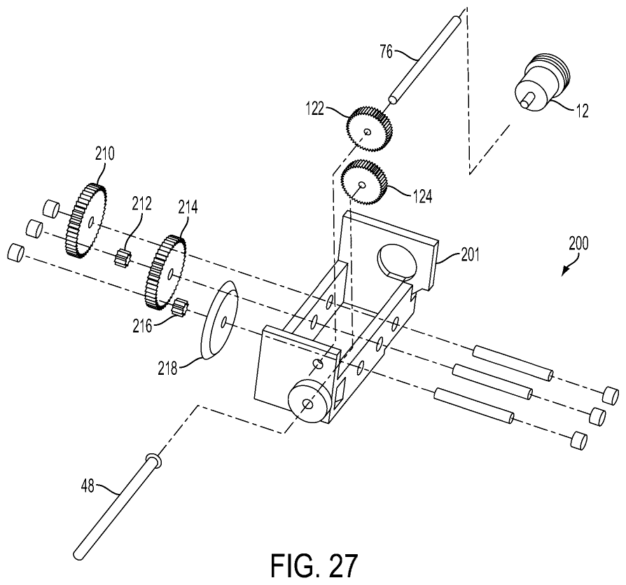

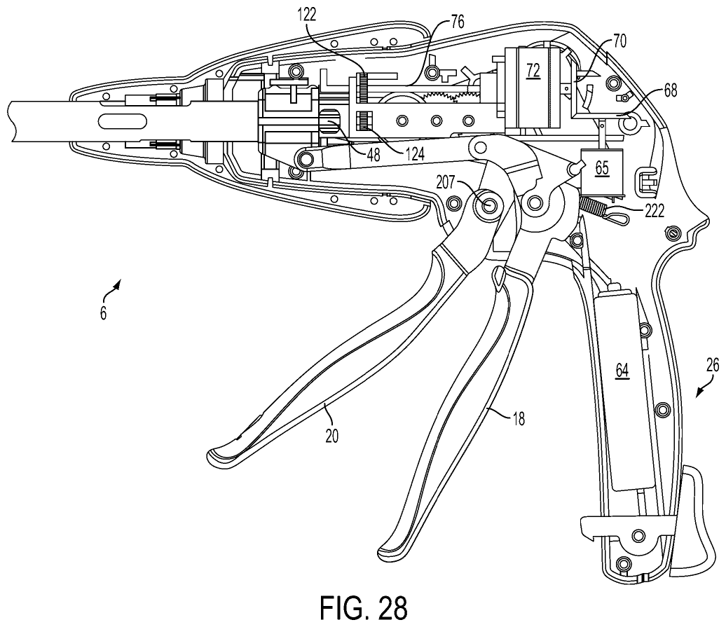

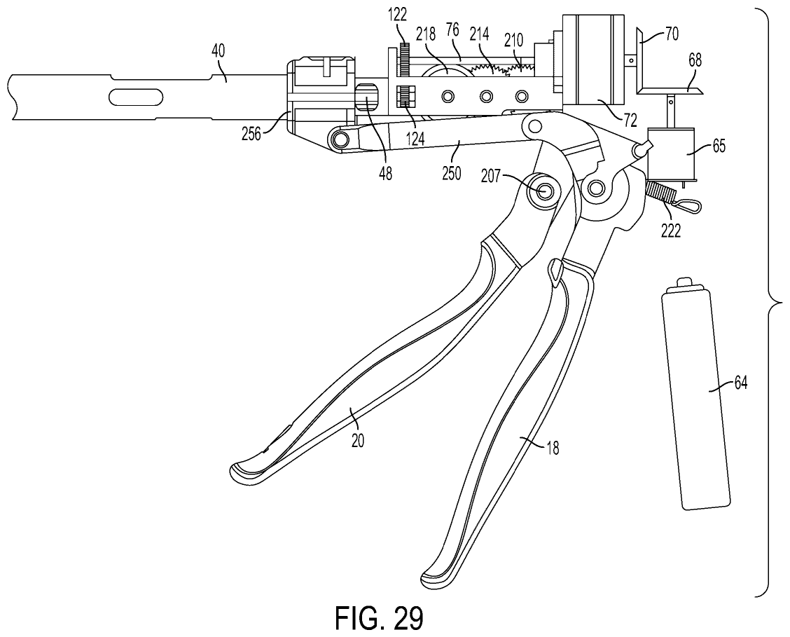

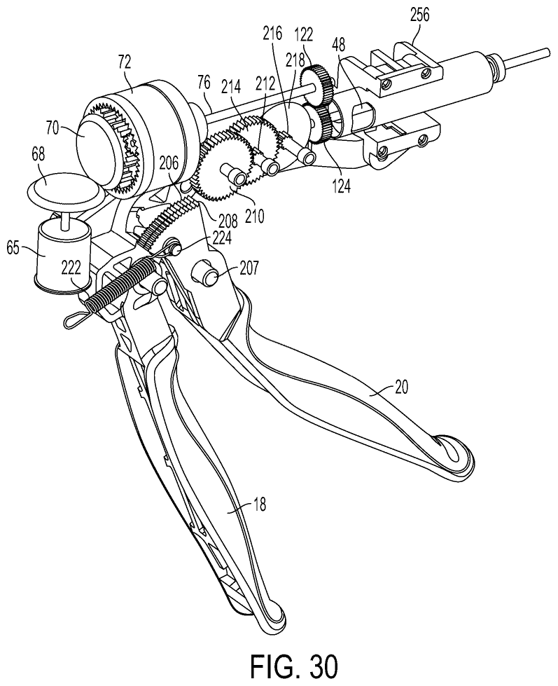

FIGS. 25-31 illustrate another embodiment of a motorized, two-stroke surgical cutting and fastening instrument 10 with power assist. The embodiment of FIGS. 25-31 is similar to that of FIGS. 6-10 except that instead of the helical gear drum 80, the embodiment of FIGS. 25-31 includes an alternative gear drive assembly. The embodiment of FIGS. 25-31 includes a gear box assembly 200 including a number of gears disposed in a frame 201, wherein the gears are connected between the planetary gear 72 and the pinion gear 124 at the proximate end of the drive shaft 48. As explained further below, the gear box assembly 200 provides feedback to the user via the firing trigger 20 regarding the deployment and loading force of the end effector 12. Also, the user may provide power to the system via the gear box assembly 200 to assist the deployment of the end effector 12. In that sense, like the embodiments described above, the embodiment of FIGS. 25-31 is another power assist, motorized instrument 10 that provides feedback to the user regarding the loading force experienced by the cutting instrument 32.

In the illustrated embodiment, the firing trigger 20 includes two pieces: a main body portion 202 and a stiffening portion 204. The main body portion 202 may be made of plastic, for example, and the stiffening portion 204 may be made out of a more rigid material, such as metal. In the illustrated embodiment, the stiffening portion 204 is adjacent to the main body portion 202, but according to other embodiments, the stiffening portion 204 could be disposed inside the main body portion 202. A pivot pin 207 may be inserted through openings in the firing trigger pieces 202, 204 and may be the point about which the firing trigger 20 rotates. In addition, a spring 222 may bias the firing trigger 20 to rotate in a CCW direction. The spring 222 may have a distal end connected to a pin 224 that is connected to the pieces 202, 204 of the firing trigger 20. The proximate end of the spring 222 may be connected to one of the handle exterior lower side pieces 59, 60.

In the illustrated embodiment, both the main body portion 202 and the stiffening portion 204 include gear portions 206, 208 (respectively) at their upper end portions. The gear portions 206, 208 engage a gear in the gear box assembly 200, as explained below, to drive the main drive shaft assembly and to provide feedback to the user regarding the deployment of the end effector 12.

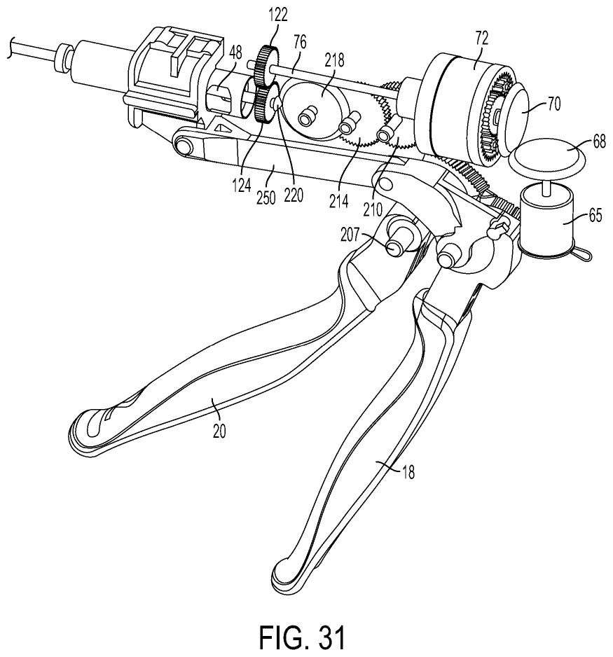

The gear box assembly 200 may include as shown, in the illustrated embodiment, six (6) gears. A first gear 210 of the gear box assembly 200 engages the gear portions 206, 208 of the firing trigger 20. In addition, the first gear 210 engages a smaller second gear 212, the smaller second gear 212 being coaxial with a large third gear 214. The third gear 214 engages a smaller fourth gear 216, the smaller fourth gear 216 being coaxial with a fifth gear 218. The fifth gear 218 is a 90° bevel gear that engages a mating 90° bevel gear 220 (best shown in FIG. 31) that is connected to the pinion gear 124 that drives the main drive shaft 48.

In operation, when the user retracts the firing trigger 20, a run motor sensor (not shown) is activated, which may provide a signal to the motor 65 to rotate at a rate proportional to the extent or force with which the operator is retracting the firing trigger 20. This causes the motor 65 to rotate at a speed proportional to the signal from the sensor. The sensor is not shown for this embodiment, but it could be similar to the run motor sensor 110 described above. The sensor could be located in the handle 6 such that it is depressed when the firing trigger 20 is retracted. Also, instead of a proportional-type sensor, an on/off type sensor may be used.

Rotation of the motor 65 causes the bevel gears 66, 70 to rotate, which causes the planetary gear 72 to rotate, which causes, via the drive shaft 76, the ring gear 122 to rotate. The ring gear 122 meshes with the pinion gear 124, which is connected to the main drive shaft 48. Thus, rotation of the pinion gear 124 drives the main drive shaft 48, which causes actuation of the cutting/stapling operation of the end effector 12.

Forward rotation of the pinion gear 124 in turn causes the bevel gear 220 to rotate, which causes, by way of the rest of the gears of the gear box assembly 200, the first gear 210 to rotate. The first gear 210 engages the gear portions 206, 208 of the firing trigger 20, thereby causing the firing trigger 20 to rotate CCW when the motor 65 provides forward drive for the end effector 12 (and to rotate CCW when the motor 65 rotates in reverse to retract the end effector 12). In that way, the user experiences feedback regarding loading force and deployment of the end effector 12 by way of the user's grip on the firing trigger 20. Thus, when the user retracts the firing trigger 20, the operator will experience a resistance related to the load force experienced by the end effector 12. Similarly, when the operator releases the firing trigger 20 after the cutting/stapling operation so that it can return to its original position, the user will experience a CW rotation force from the firing trigger 20 that is generally proportional to the reverse speed of the motor 65.

It should also be noted that in this embodiment the user can apply force (either in lieu of or in addition to the force from the motor 65) to actuate the main drive shaft assembly (and hence the cutting/stapling operation of the end effector 12) through retracting the firing trigger 20. That is, retracting the firing trigger 20 causes the gear portions 206, 208 to rotate CCW, which causes the gears of the gear box assembly 200 to rotate, thereby causing the pinion gear 124 to rotate, which causes the main drive shaft 48 to rotate.

Although not shown in FIGS. 25-31, the instrument 10 may further include reverse motor and stop motor sensors. As described above, the reverse motor and stop motor sensors may detect, respectively, the end of the cutting stroke (full deployment of the knife 32 and sled 33) and the end of retraction operation (full retraction of the knife 32). A circuit similar to that described above in connection with FIG. 11 may be used to appropriately power the motor 65.

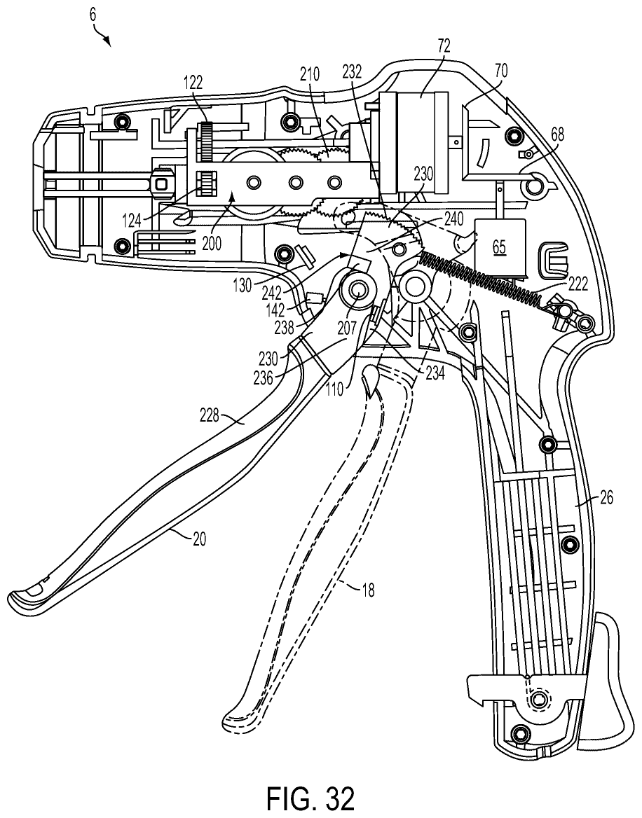

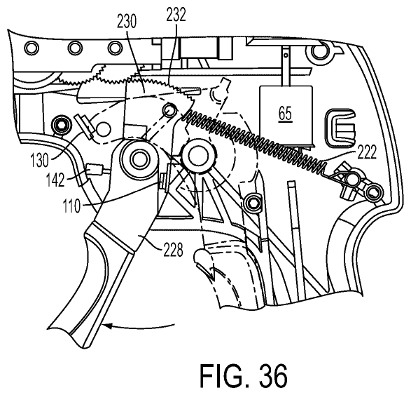

FIGS. 32-36 illustrate another embodiment of a two-stroke, motorized surgical cutting and fastening instrument 10 with power assist. The embodiment of FIGS. 32-36 is similar to that of FIGS. 25-31 except that in the embodiment of FIGS. 32-36, the firing trigger 20 includes a lower portion 228 and an upper portion 230. Both portions 228, 230 are connected to and pivot about a pivot pin 207 that is disposed through each portion 228, 230. The upper portion 230 includes a gear portion 232 that engages the first gear 210 of the gear box assembly 200. The spring 222 is connected to the upper portion 230 such that the upper portion is biased to rotate in the CW direction. The upper portion 230 may also include a lower arm 234 that contacts an upper surface of the lower portion 228 of the firing trigger 20 such that when the upper portion 230 is caused to rotate CW the lower portion 228 also rotates CW, and when the lower portion 228 rotates CCW the upper portion 230 also rotates CCW. Similarly, the lower portion 228 includes a rotational stop 238 that engages a lower shoulder of the upper portion 230. In that way, when the upper portion 230 is caused to rotate CCW the lower portion 228 also rotates CCW, and when the lower portion 228 rotates CW the upper portion 230 also rotates CW.

The illustrated embodiment also includes the run motor sensor 110 that communicates a signal to the motor 65 that, in various embodiments, may cause the motor 65 to rotate at a speed proportional to the force applied by the operator when retracting the firing trigger 20. The sensor 110 may be, for example, a rheostat or some other variable resistance sensor, as explained herein. In addition, the instrument 10 may include a reverse motor sensor 130 that is tripped or switched when contacted by a front face 242 of the upper portion 230 of the firing trigger 20. When activated, the reverse motor sensor 130 sends a signal to the motor 65 to reverse direction. Also, the instrument 10 may include a stop motor sensor 142 that is tripped or actuated when contacted by the lower portion 228 of the firing trigger 20. When activated, the stop motor sensor 142 sends a signal to stop the reverse rotation of the motor 65.

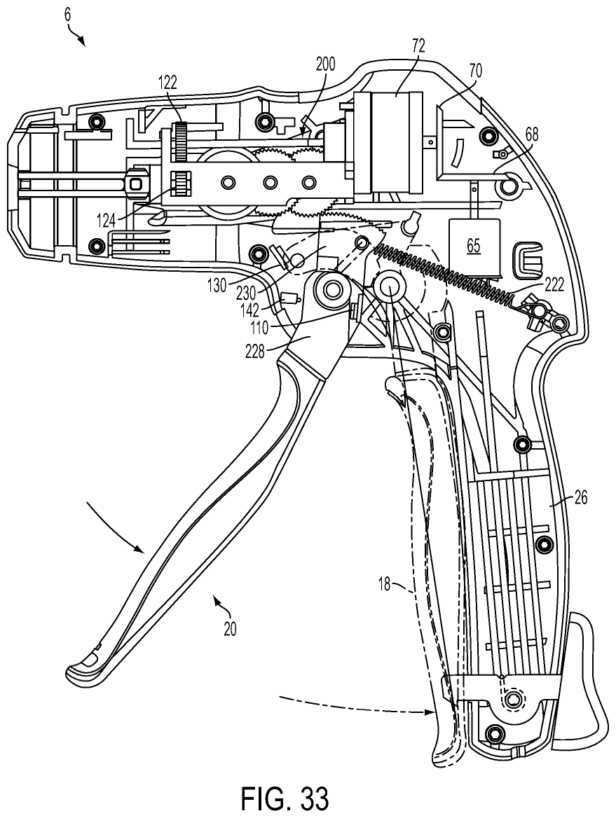

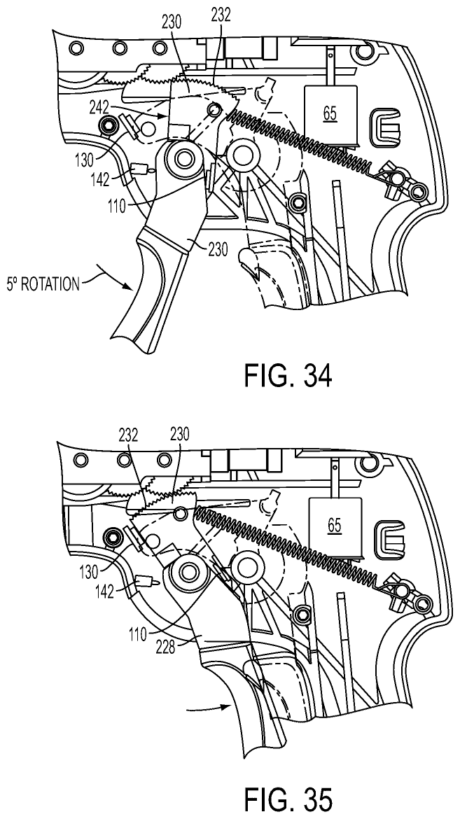

In operation, when an operator retracts the closure trigger 18 into the locked position, the firing trigger 20 is retracted slightly (through mechanisms known in the art, including U.S. Pat. No. 6,978,921, which issued Dec. 27, 2005, entitled SURGICAL STAPLING INSTRUMENT INCORPORATING AN E-BEAM FIRING MECHANISM and U.S. Pat. No. 6,905,057, which issued Jun. 14, 2005, entitled SURGICAL STAPLING INSTRUMENT INCORPORATING A FIRING MECHANISM HAVING A LINKED RACK TRANSMISSION, both of which are incorporated herein by reference) so that the user can grasp the firing trigger 20 to initiate the cutting/stapling operation, as shown in FIGS. 32 and 33. At that point, as shown in FIG. 33, the gear portion 232 of the upper portion 230 of the firing trigger 20 moves into engagement with the first gear 210 of the gear box assembly 200. When the operator retracts the firing trigger 20, according to various embodiments, the firing trigger 20 may rotate a small amount, such as five degrees, before tripping the run motor sensor 110, as shown in FIG. 34. Activation of the sensor 110 causes the motor 65 to forward rotate at a rate proportional to the retraction force applied by the operator. The forward rotation of the motor 65 causes, as described above, the main drive shaft 48 to rotate, which causes the knife 32 in the end effector 12 to be deployed (e.g., begin traversing the channel 22). Rotation of the pinion gear 124, which is connected to the main drive shaft 48, causes the gears 210-220 in the gear box assembly 200 to rotate. Since the first gear 210 is in engagement with the gear portion 232 of the upper portion 230 of the firing trigger 20, the upper portion 230 is caused to rotate CCW, which causes the lower portion 228 to also rotate CCW.

When the knife 32 is fully deployed (e.g., at the end of the cutting stroke), the front face 242 of the upper portion 230 trips the reverse motor sensor 130, which sends a signal to the motor 65 to reverse rotational direction. This causes the main drive shaft assembly to reverse rotational direction to retract the knife 32. Reverse rotation of the main drive shaft assembly causes the gears 210-220 in the gear box assembly to reverse direction, which causes the upper portion 230 of the firing trigger 20 to rotate CW, which causes the lower portion 228 of the firing trigger 20 to rotate CW until the front face 242 of the upper portion 230 trips or actuates the stop motor sensor 142 when the knife 32 is fully retracted, which causes the motor 65 to stop. In that way, the user experiences feedback regarding deployment of the end effector 12 by way of the user's grip on the firing trigger 20. Thus, when the user retracts the firing trigger 20, the operator will experience a resistance related to the deployment of the end effector 12 and, in particular, to the loading force experienced by the knife 32. Similarly, when the operator releases the firing trigger 20 after the cutting/stapling operation so that it can return to its original position, the user will experience a CW rotation force from the firing trigger 20 that is generally proportional to the reverse speed of the motor 65.

It should also be noted that in this embodiment the user can apply force (either in lieu of or in addition to the force from the motor 65) to actuate the main drive shaft assembly (and hence the cutting/stapling operation of the end effector 12) through retracting the firing trigger 20. That is, retracting the firing trigger 20 causes the gear portion 232 of the upper portion 230 to rotate CCW, which causes the gears of the gear box assembly 200 to rotate, thereby causing the pinion gear 124 to rotate, which causes the main drive shaft assembly to rotate.

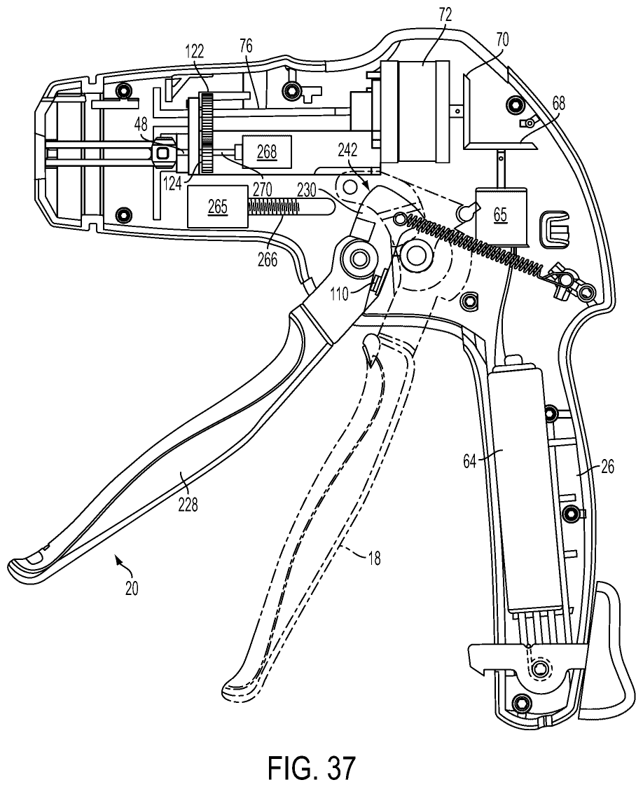

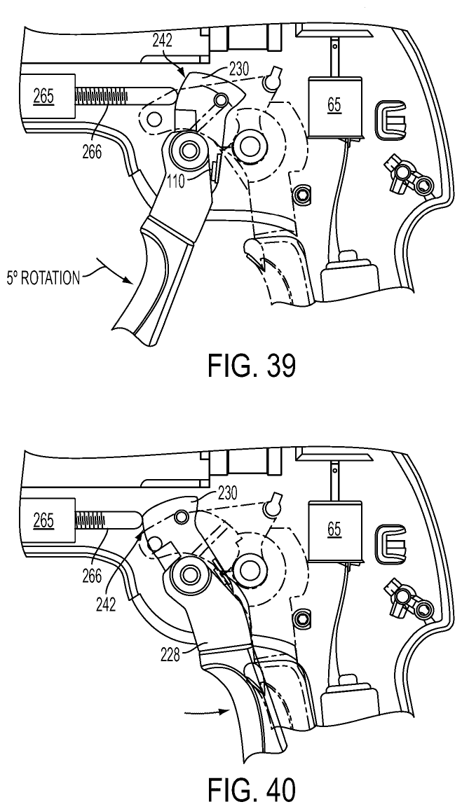

The above-described embodiments employed power-assist user feedback systems, with or without adaptive control (e.g., using a sensor 110, 130, and 142 outside of the closed loop system of the motor, gear drive train, and end effector) for a two-stroke, motorized surgical cutting and fastening instrument. That is, force applied by the user in retracting the firing trigger 20 may be added to the force applied by the motor 65 by virtue of the firing trigger 20 being geared into (either directly or indirectly) the gear drive train between the motor 65 and the main drive shaft 48. In other embodiments, the user may be provided with tactile feedback regarding the position of the knife 32 in the end effector 12, but without having the firing trigger 20 geared into the gear drive train. FIGS. 37-40 illustrate one embodiment of a motorized surgical cutting and fastening instrument 10 with such a tactile position feedback system.

In the illustrated embodiment of FIGS. 37-40, the firing trigger 20 may have a lower portion 228 and an upper portion 230, similar to the instrument 10 shown in FIGS. 32-36. Unlike the embodiment of FIGS. 32-36, however, the upper portion 230 does not have a gear portion that mates with part of the gear drive train. Instead, the instrument 10 includes a second motor 265 with a threaded rod 266 threaded therein. The threaded rod 266 reciprocates longitudinally in and out of the motor 265 as the motor 265 rotates, depending on the direction of rotation. The instrument 10 also includes an encoder 268 that is responsive to the rotations of the main drive shaft 48 for translating the incremental angular motion of the main drive shaft 48 (or other component of the main drive assembly) into a corresponding series of digital signals, for example. In the illustrated embodiment, the pinion gear 124 includes a proximate drive shaft 270 that connects to the encoder 268.

The instrument 10 also includes a control circuit (not shown), which may be implemented using a microcontroller or some other type of integrated circuit, that receives the digital signals from the encoder 268. Based on the signals from the encoder 268, the control circuit may calculate the stage of deployment of the knife 32 in the end effector 12. That is, the control circuit can calculate if the knife 32 is fully deployed, fully retracted, or at an intermittent stage. Based on the calculation of the stage of deployment of the end effector 12, the control circuit may send a signal to the second motor 265 to control its rotation to thereby control the reciprocating movement of the threaded rod 266.

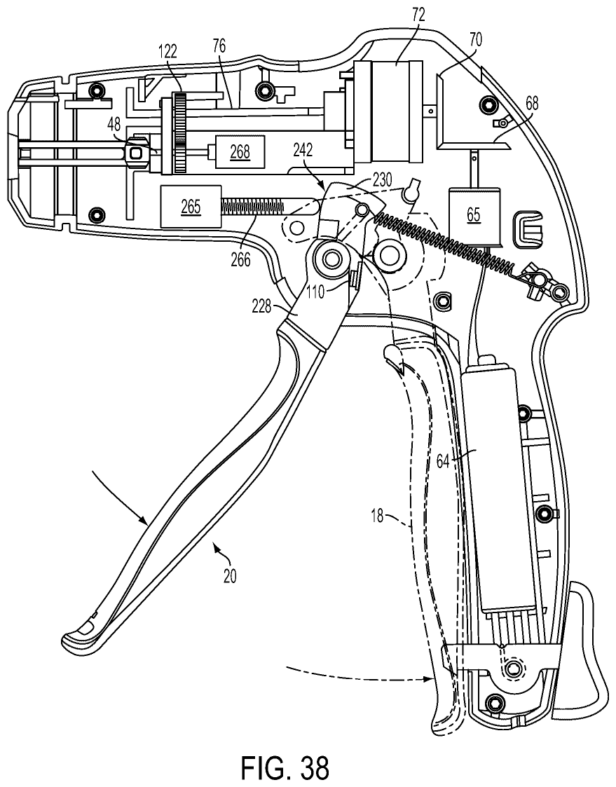

In operation, as shown in FIG. 37, when the closure trigger 18 is not locked into the clamped position, the firing trigger 20 rotated away from the pistol grip portion 26 of the handle 6 such that the front face 242 of the upper portion 230 of the firing trigger 20 is not in contact with the proximate end of the threaded rod 266. When the operator retracts the closure trigger 18 and locks it in the clamped position, the firing trigger 20 rotates slightly towards the closure trigger 18 so that the operator can grasp the firing trigger 20, as shown in FIG. 38. In this position, the front face 242 of the upper portion 230 contacts the proximate end of the threaded rod 266.

As the user then retracts the firing trigger 20, after an initial rotational amount (e.g., 5 degrees of rotation) the run motor sensor 110 may be activated such that, as explained above, the sensor 110 sends a signal to the motor 65 to cause it to rotate at a forward speed proportional to the amount of retraction force applied by the operator to the firing trigger 20. Forward rotation of the motor 65 causes the main drive shaft 48 to rotate via the gear drive train, which causes the knife 32 and sled 33 to travel down the channel 22 and sever tissue clamped in the end effector 12. The control circuit receives the output signals from the encoder 268 regarding the incremental rotations of the main drive shaft assembly and sends a signal to the second motor 265 to cause the second motor 265 to rotate, which causes the threaded rod 266 to retract into the motor 265. This allows the upper portion 230 of the firing trigger 20 to rotate CCW, which allows the lower portion 228 of the firing trigger to also rotate CCW. In that way, because the reciprocating movement of the threaded rod 266 is related to the rotations of the main drive shaft assembly, the operator of the instrument 10, by way of his/her grip on the firing trigger 20, experiences tactile feedback as to the position of the end effector 12. The retraction force applied by the operator, however, does not directly affect the drive of the main drive shaft assembly because the firing trigger 20 is not geared into the gear drive train in this embodiment.

By virtue of tracking the incremental rotations of the main drive shaft assembly via the output signals from the encoder 268, the control circuit can calculate when the knife 32 is fully deployed (e.g., fully extended). At this point, the control circuit may send a signal to the motor 65 to reverse direction to cause retraction of the knife 32. The reverse direction of the motor 65 causes the rotation of the main drive shaft assembly to reverse direction, which is also detected by the encoder 268. Based on the reverse rotation detected by the encoder 268, the control circuit sends a signal to the second motor 265 to cause it to reverse rotational direction such that the threaded rod 266 starts to extend longitudinally from the motor 265. This motion forces the upper portion 230 of the firing trigger 20 to rotate CW, which causes the lower portion 228 to rotate CW. In that way, the operator may experience a CW force from the firing trigger 20, which provides feedback to the operator as to the retraction position of the knife 32 in the end effector 12. The control circuit can determine when the knife 32 is fully retracted. At this point, the control circuit may send a signal to the motor 65 to stop rotation.

According to other embodiments, rather than having the control circuit determine the position of the knife 32, reverse motor and stop motor sensors may be used, as described above. In addition, rather than using a proportional sensor 110 to control the rotation of the motor 65, an on/off switch or sensor can be used. In such an embodiment, the operator would not be able to control the rate of rotation of the motor 65. Rather, it would rotate at a preprogrammed rate.

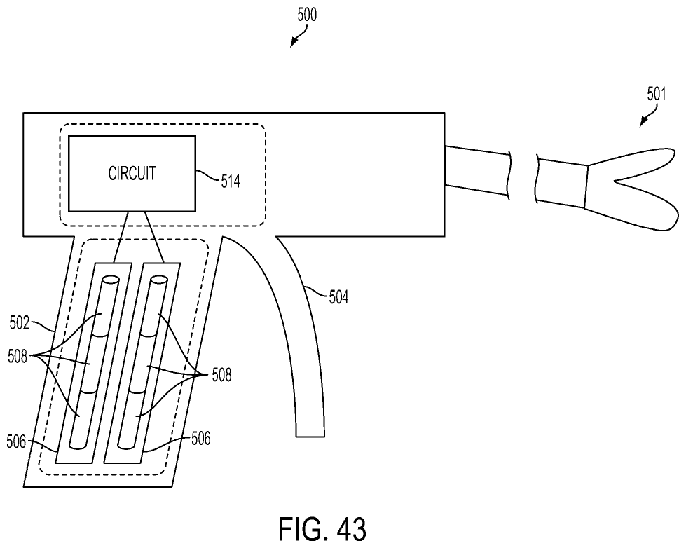

FIGS. 43-61 herein describe embodiments of batteries and battery configurations for use with powered surgical devices. The batteries and battery configurations described below may be utilized with any suitable powered surgical instrument including for example, the instrument embodiments described above. In addition to or instead of the functionality of the embodiments described herein above surgical instruments utilizing the batteries and battery configurations of FIGS. 43-61 may comprise end effectors for cutting, clasping, laser cutting and/or coagulation, RF cutting and/or coagulation, ultrasonic cutting and/or coagulation, etc. Additional details regarding surgical instruments and battery units are described in U.S. patent application Ser. No. 12/884,995, entitled, POWER CONTROL ARRANGEMENTS FOR SURGICAL INSTRUMENTS AND BATTERIES, filed Sep. 17, 2010, now U.S. Pat. No. 8,632,525, which is incorporated herein by reference in its entirety.

FIG. 43 illustrates one embodiment of a surgical instrument 500 comprising a pair of asymmetrically-shaped battery packs 506. The instrument 500 may comprise a handle 502, a trigger 504 and an end effector 501. According to various embodiments, the handle 502, trigger 504 and end effector 501 may operate in a manner similar to that of the various handles 6, triggers, 18, 20 and end effectors 12 described herein. In addition to or instead of the functionality described herein above, the end effector 501 may comprise surgical implements for cutting, clasping, laser cutting and/or coagulation, RF cutting and/or coagulation, ultrasonic cutting and/or coagulation, etc.

The handle 502 of the instrument 502 may house battery packs 506, as shown. The battery packs 506 may be electrically connected to and may provide power to a circuit 514 of the instrument 500. The circuit may be located in the handle 502, as shown, in the end effector 501, or in any combination of locations within the instrument 500. In use, the circuit 514 may power the operation of at least one surgical implement at the end effector 501. For example, the circuit 514 may comprise an electric motor for operating an electrically powered cutter, clasper, or other mechanical device. In addition to, or instead of a motor, the circuit 514 may comprise suitable circuit components for implementing an RF, ultrasonic, or other type of non-motor-powered surgical implement.

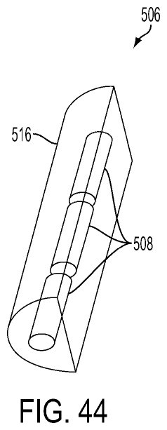

FIG. 44 illustrates one embodiment of a battery pack 506 outside of the handle 502. The battery pack 506 may have an asymmetric cross-sectional shape. For example, in the embodiment pictured in FIG. 44, the battery pack 506 has a half-ovaloid shape. It will be appreciated that other asymmetric cross-sectional shapes could be used. As illustrated, the battery pack 506 comprises three cells 508. The cells 508 may be any suitable type of cell including, for example, lithium-ion cells such as the CR123-type cell and/or the CR2-type cell. The cells 508 may be electrically connected to one another in series or parallel. The number of cells 508 may be chosen to the power of any accidental discharge from the battery pack 506. For example, the number of connected cells 508 may be selected such that the cumulative energy available to an arc or short is less than the energy required to ignite common shipping and/or packing materials. According to various embodiments, this value may be defined by appropriate government regulations.

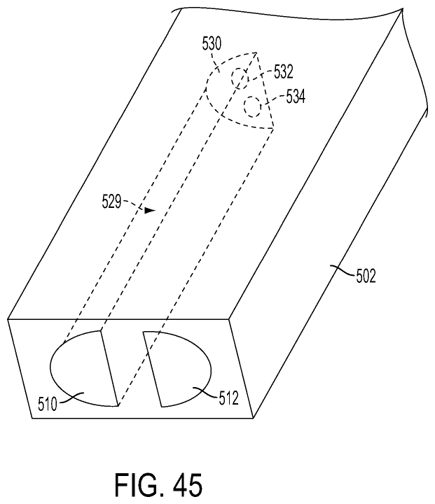

FIG. 45 illustrates one embodiment of the handle 502 illustrating cavities 510, 512 for receiving the battery packs 506. The cavities 510, 512 may have an asymmetric cross-sectional shape corresponding to the cross-sectional shape of the battery packs 506. This may allow the battery packs 506 to be received within the cavities 510, 512, as illustrated in FIG. 43. An interior portion 529 of the cavity 510 is also shown in FIG. 45. A wall 530 may comprise contacts 532, 534. The contacts 532, 534 may be connected to the circuit 514 and may be configured to connect the battery pack 506 to the circuit 514 when the battery pack 506 is installed in the cavity 510. It will be appreciated that cavity 512 may comprise a similar interior portion and similar contacts. Illustration of these elements is omitted in FIG. 45, however, for clarity.

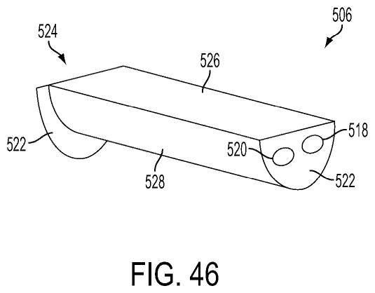

FIG. 46 illustrates one embodiment of the battery pack of 506 showing positive electrode contact 518 and negative electrode contact 520. Upon insertion of the battery pack 506 within the cavity 510, the electrode contacts 518 and 520 may connect to the contacts 532, 534, illustrated in FIG. 45, to establish a connection between the battery pack 506 and the circuit 514. The electrode contacts 518, 520 are illustrated on a first end 522 of the battery pack 506. It will be appreciated, however, that the electrode contacts 518, 520 may be positioned on any other surface of the battery pack 506 including, for example, the end 524, flat face 526 and/or curved face 528. Accordingly, the contacts 532, 534 may be positioned on a corresponding surface of the interior portion 529 of the cavity 510.

The asymmetric cross sectional shape of the battery packs 506 and the cavities 510, 512 may ensure that the battery packs 506 are inserted into the instrument 500 with the correct polarity. For example, due to its asymmetric cross-sectional shape, the end 522 of the battery pack 506 may fit into the cavity 510 of the handle 502 in only one orientation, ensuring that the correct electrodes 518, 520, 532, 534 are in contact with one another. Similarly, the end 522 of the battery pack 506 may fit into the cavity 512 in only one orientation. Because the cross-sectional shape of the cavity 512 is reciprocal to that of the cavity 510, the orientation of the electrode contacts 518, 520 may be reversed in the cavity 512 relative to the cavity 510. Accordingly, when the cavities 510, 512 have reciprocal cross-sectional shapes, as illustrated, the position of the contacts (not shown) within the cavity 512 may also be reversed to ensure correct polarity.

The clinician may be relied upon to recognize that the end 522 of the battery pack 506 with the electrodes 518, 520 is properly inserted into the cavities 510, 512. According to various embodiments, however, the form of the battery pack 506 may be manipulated to make it difficult or impossible for the end 524 of the battery pack 506 to be inserted into the cavities 510, 512. For example, in FIG. 46, the battery pack 506 is shown with an optional flange 522 at the end 524. The flange 522 may extend beyond the battery pack 506 to ensure that the end 524 cannot be inserted into one of the cavities 510, 512. Although the instrument 500 illustrated utilizes two battery packs 506 and defines two cavities 510, 512, it will be appreciated that more or additional battery packs and corresponding cavities may be used.

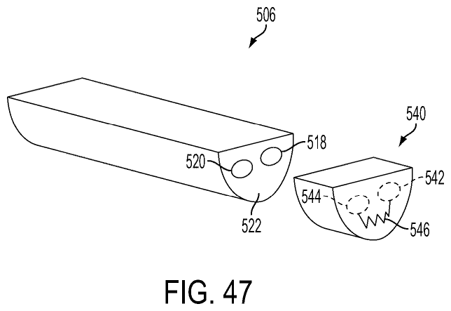

FIG. 47 illustrates one embodiment of the battery pack 506 in conjunction with a discharge plug 540. The discharge plug 540 may be attached to the end 522 of the battery pack 506, for example, after use of the battery pack 506 is complete. In certain embodiments, the discharge plug 540 may have a cross-sectional area slightly larger than that of the battery pack 506 and may slide over the end 522. The discharge plug may comprise electrode contacts 542, 546 electrically connected to one another via a resistive element 546. The resistive element 546 may be any suitable resistive element having any suitable electrical resistance and/or impedance. With the discharge in place, the electrode contacts 542, 546 may contact positive and negative electrode contacts 518, 520. This may place the resistive element 546 in series with the battery pack 506, causing the battery to drain. In this way, the battery pack 506 may be drained either prior to or during disposal, reducing hazard disposal.

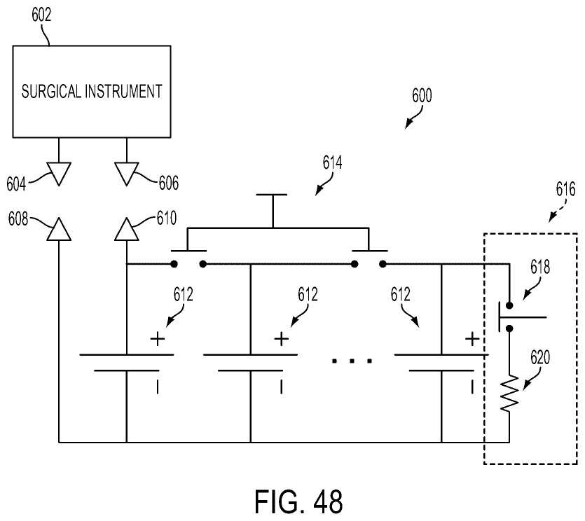

FIG. 48 illustrates a schematic diagram of one embodiment of a surgical instrument 602 and a battery pack 600. The surgical instrument 602 may operate in a manner similar to that of the surgical instruments 10, 500 described herein above. For example, the instrument 602 may be any suitable type of surgical instrument utilizing battery power including, for example, instruments having motorized implements for cutting, motorized implements for stapling, RF implements for cutting and/or coagulating, ultrasonic implements for cutting and/or coagulating, laser implements for cutting/coagulating, etc. The surgical instrument 602 may comprise a pair of electrodes 604, 606, which, when the battery pack 600 is connected to the surgical instrument 603, may connect with a pair of electrodes 608, 610 of the battery pack 600.

The battery pack 600 may comprise a plurality of cells 612. The cells 612 may be any suitable type of cell. According to various embodiments, the cells may be lithium-ion cells such as the CR123-type cell and/or the CR2-type cell. A switch 614 may have an open position and a closed position. The switch 614 may be any suitable type of mechanical or solid state switch. When the switch 614 is in the open position, the cells 612 may be electrically disconnected from one another. When the switch 614 is in the closed position, the cells 612 may be electrically connected to one another. For example, in FIG. 48, the cells 612 are shown connected in parallel. In various embodiments, however, the cells 612 may be connected in series or in any other desirable configuration. The switch 614 may be engaged to the closed position at the time that the battery pack 600 is connected to the surgical instrument 602. For example, the switch 614 may be manually engaged by a clinician using the surgical instrument 602 either before or after the battery pack 600 is connected to the instrument 602. Also, according to various embodiments, the switch 614 may be engaged to the closed position automatically when the battery pack 600 is connected to the instrument 602 (e.g., by placing at least a portion of the battery pack 600 within the surgical instrument 602).

The battery pack 600 may also comprise a discharge system 616. The discharge system 616 may comprise a discharge switch 618 and a resistive element 620. The resistive element 620 may be any suitable resistive element having any suitable electrical resistance and/or impedance. The discharge switch 618 may have an open position and a closed position. When the discharge switch is in the open position, the resistive element 620 may not be electrically connected to the battery pack. When the discharge switch 618 is in the closed position, the resistive element 620 may be electrically connected across the cells 612 of the battery pack 600. In this way, the cells 612 may drain when the discharge switch is closed 618. The discharge switch 620 may be any type of mechanical or solid state switch. The discharge switch 618 may be manually or automatically transitioned from the open to the closed position, for example, upon installation of the battery pack 614 to the instrument 602 or upon removal of the surgical instrument 602 from the instrument 602. In some embodiments, the cells 612 may deliver sufficient power and/or the resistive element 620 may be designed such that discharge switch 618 may be closed while the instrument 602 is in use.

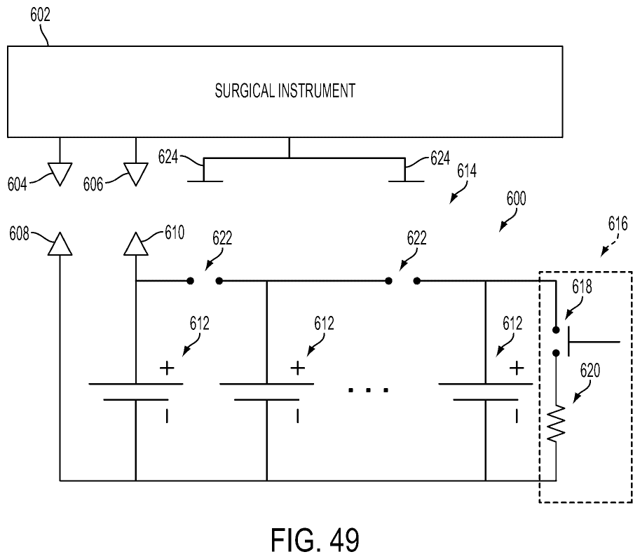

FIG. 49 illustrates an alternate embodiment of the battery pack 600 and surgical instrument 602 shown in FIG. 48. As illustrated in FIG. 49, the switch 614 may comprise at least one open portion 622 and at least one contactor 624. As illustrated, the at least one contactor 624 may be a part of the surgical instrument 602. In this way, the cells 612 may be electrically connected to one another when the battery pack 600 is installed to the surgical instrument 602, bringing the at least one connector portion in electrical contact with the at least one open portion 622.

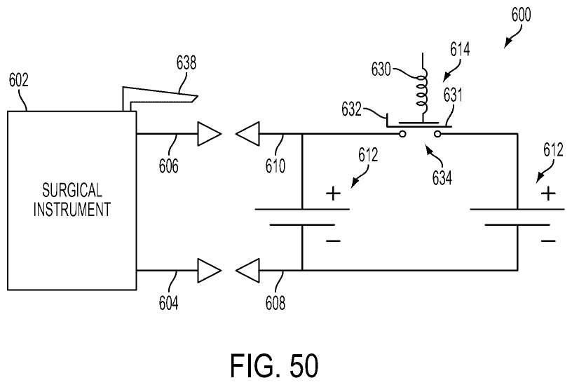

FIG. 50 illustrates another embodiment of the battery pack 600 of FIG. 48. As illustrated in FIG. 50, the switch 614, is implemented with an open portion 634, a contactor 636 and a movable tab 631. The contactor 636 may be mechanically biased against the open portion 634, for example, by a spring 630. The movable tab 631 may be positioned between the open portion 634 and the contactor 636. The movable tab 631 may be made from an insulating material, such as plastic. In this way, the cells 612 may not be electrically connected to one another when the movable tab 631 is in place. When the battery pack 600 is ready for use, the tab 631 may be removed, for example, by the clinician. When the tab 631 is removed, the contactor 636 may be mechanically pushed into electrical contact with the open portion 634, resulting in the electrical connection of the cells 612 to one another. According to various embodiments, the tab 631 may comprise a portion 632 configured to be received by a corresponding portion 638 of the surgical instrument. When the battery pack 600 is installed to the instrument, the portion 638 of the surgical instrument 602 may contact the tab 631, tending to remove it from between the open portion 634 and the contactor 636. The tab 631 may be made from a polymer or any suitable electrically insulating material. Also, according to various embodiments, the tab 631 may have a thickness of about 1 mil.

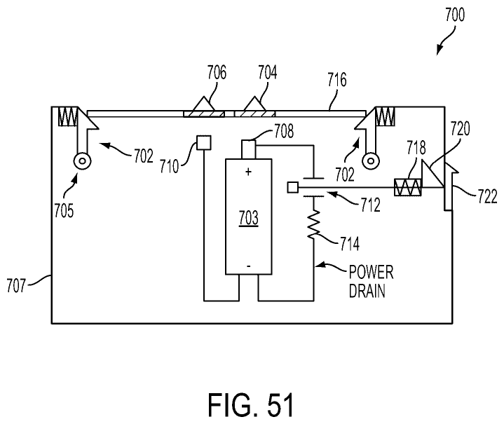

FIGS. 51-53 illustrate one mechanical embodiment of a battery pack 700 implementing the schematic of the battery pack 600 shown in FIG. 48. The battery pack 700 comprises a casing 707 having therein a battery 703 comprising a plurality of cells that can be interconnected to one another by connecting contacts 708, 710. A discharge switch 712 may, when in the closed position, connect a resistive element 714 across the terminals of the cells 703, causing them to discharge. The battery pack 700 may comprise a pair of contacts 706, 704 positioned on a switch platform 716. The contactors 706, 704 may have an open position shown in FIG. 51 and a closed position. In the closed position, the contactors 706, 704 may be placed in electrical contact with the contacts 708, 710, causing the cells 703 to be interconnected to one another. Collectively, the switch platform 716, contacts 708, 710, and contactors 704, 706 may form a switch. According to various embodiment, when the switch is closed (e.g., the contactors 704, 706 are in contact with the contacts 708, 710), the cells of the battery 703 may electrically interconnected.

The switch platform 716 may be coupled to a clutch 705 comprising a pair of locking mechanisms 702. In the position shown in FIG. 51, the clutch (including locking mechanisms 702) is engaged, holding the switch platform 716 in the open position. The battery pack 700 may also comprise a discharge switch 712. In a closed position, the discharge switch 712 may switch a resistive element 714 across the anode and the cathode of the cells 703, causing the cells to discharge. As illustrated in FIG. 51, the discharge switch may be mechanically biased to the closed position by a spring 718. The bias of the spring 718, however, may be overcome by a stopper 720 in contact with a movable portion or panel 722 of the casing 707.

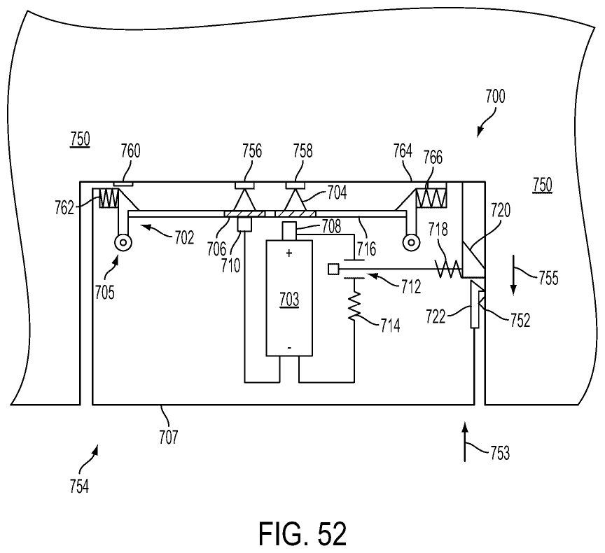

FIG. 52 illustrates a configuration of the battery pack 700 of FIG. 51 upon insertion into a surgical instrument 750, illustrated in cross-section. The battery pack 700 may be inserted into a cavity 754 defined by the instrument 750. The cavity 754 may be positioned at any portion of the instrument 750 including, in various embodiments, at a handle portion. The cavity 754 may comprise a pair of contacts 756, 758 that may be aligned with contactors 706, 708. The battery pack 700 may be inserted into the instrument 750 in the direction of arrow 753. As the battery pack 700 is inserted, contactors 706, 704 may come into contact with the contacts 756, 758. This may force the contactors 706, 708, and the switch platform 716 toward the contacts 708, 710 such that the contactors 706, 704 are in electrical communication with the contacts 710, 708 and the contacts 756, 758, which may cause the cells of the battery 703 to be interconnected and connected to the instrument 750.

According to various embodiments, pressure from the contacts 756, 758 may overcome the force of the clutch 705, disengaging the lock mechanisms 702, allowing the switch platform 716 to translate towards the contacts 708, 710. Also, in various embodiments, a portion of an interior of the cavity 754 may comprise one or more keyed portions 760, 764 that are aligned with one or more receptacles 762, 766 associated (e.g., mechanically or electronically) with the lock mechanisms 702. When the keyed portions 760, 764 come into contact with the receptacles 762, 766, the clutch 705 lock mechanisms 702 may be enabled to disengage, allowing the switch platform 716 to assume the position illustrated in FIG. 52. According to various embodiments, after the switch platform 716 assumes the position illustrated in FIG. 52, the lock mechanisms 702 may re-engage, locking the switch platform 716 in place. According to various embodiments, this may make it difficult for the battery pack 700 to lose electrical connectivity with the instrument 750 after insertion.

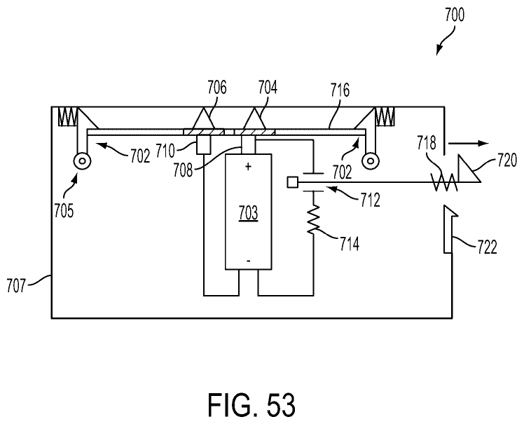

The interior of the cavity 754 may also comprise a feature 752 (e.g., an extension), for contacting the panel 722. For example, as the battery pack 700 is inserted into the cavity 754, the extension 752 may contact the panel 722, sliding it in the direction of arrow 755 and allowing the stopper 720 to protrude through the casing 707 (e.g., because of the biasing of the spring 718). According to various embodiments, the stopper 720 may contact the interior wall of the cavity 754, preventing the discharge switch 712 from being closed. FIG. 53 illustrated one embodiment of the battery pack 700 after removal from the surgical instrument 750. The switch platform 716 may be locked by the lock mechanisms 702 into the same position shown in FIG. 52. Also, within the interior wall of the cavity 754, the stopper 720 may protrude from the casing 707 by an amount suitable to close the discharge switch 712. This may cause the battery 703 to discharge.

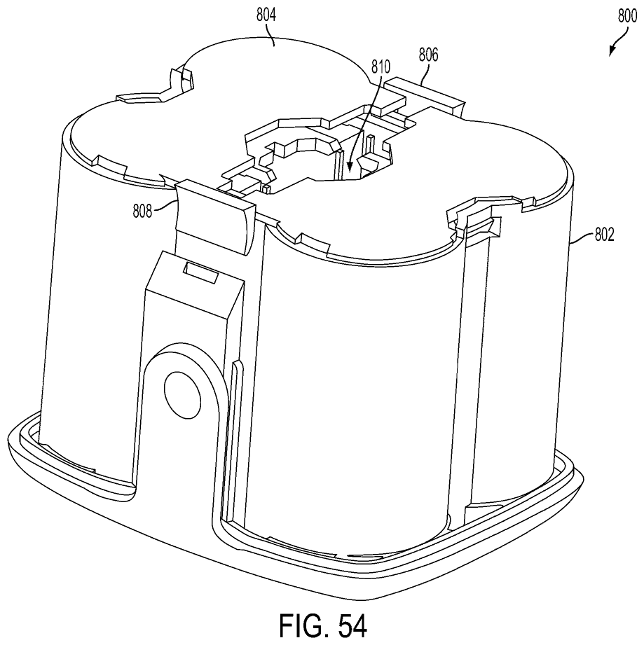

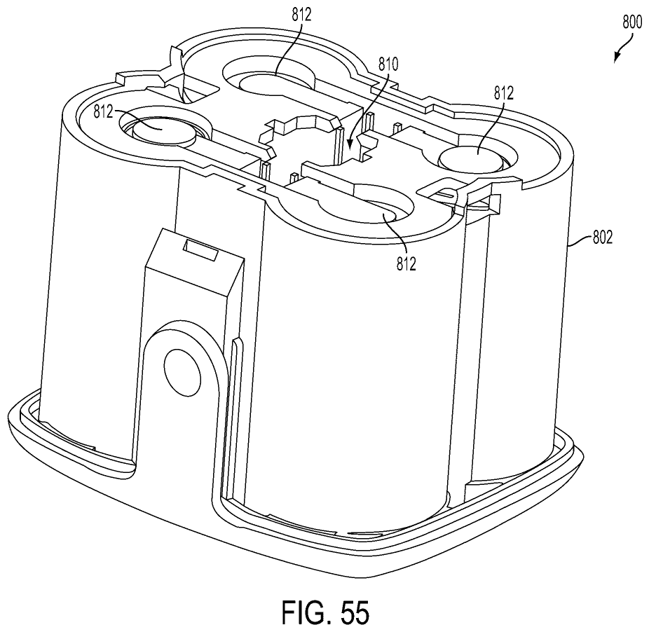

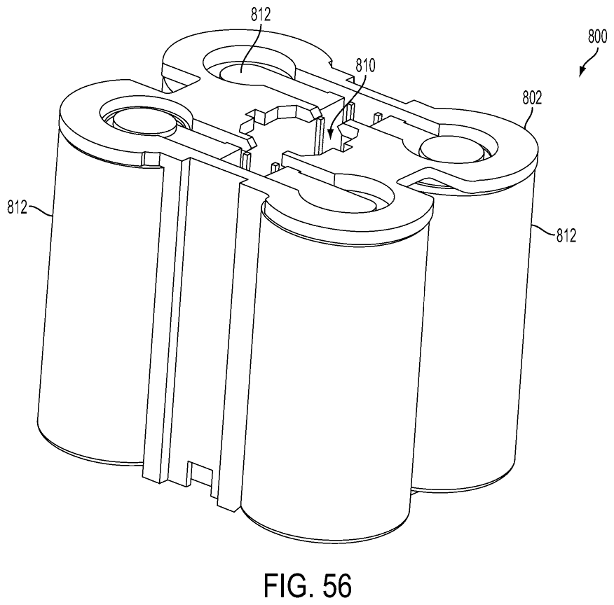

FIGS. 54-61 illustrate another mechanical embodiment of a battery pack 800 implementing the schematic of the battery pack 600 shown in FIG. 48. The battery pack 800 may comprise a casing 802 defining an interior cavity 810. The casing 802 may be covered by a cap 804 that may be secured to the casing 802 utilizing one or more mechanical latches 806, 808. FIG. 55 illustrates one embodiment of the battery pack 800 with the cap 804 removed to show a plurality of cells 812 within. Any suitable number and/or type of cells 812 may be used. For example, CR123 and/or CR2 cells may be used. FIG. 56 illustrates one embodiment of the battery pack 800 with a portion of the casing 802 removed to reveal the cells 812.

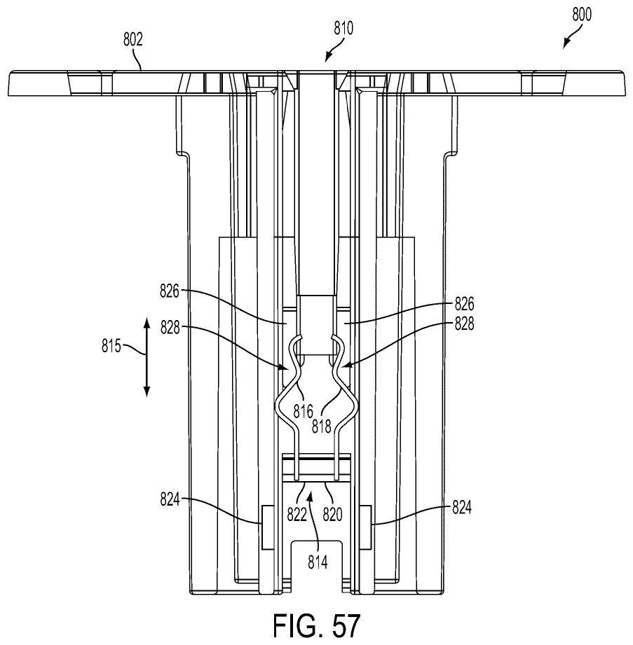

FIG. 57 illustrates a cross-sectional view of one embodiment of the battery pack 800 including a battery drain 814. The battery drain 814 may be positioned within the interior cavity 810 and may be slidable within the interior cavity 810 in the directions of arrow 815. The drain 814 may comprise at least two contacts 818, 816. A portion of the contacts 818, 816 may touch wall 826 of the interior cavity 810. According to various embodiments, the contacts 816, 818 may be biased to exert a force against the walls 826 in order to resist movement of the drain 814 in the direction of the arrows 815. Also, in some embodiments, the walls 826 may define one or more protrusions or catch members 828 shaped to be received by a portion of one or more of the contacts 816, 818 to hold the drain 814 at a first position, as shown in FIG. 57. Additionally, the walls 826 may define one or more electrodes 824. The electrodes 824 may be wired to the cells 812, such that making an electrical connection across the electrodes 824 may short the positive and negative electrodes of the cells 812.

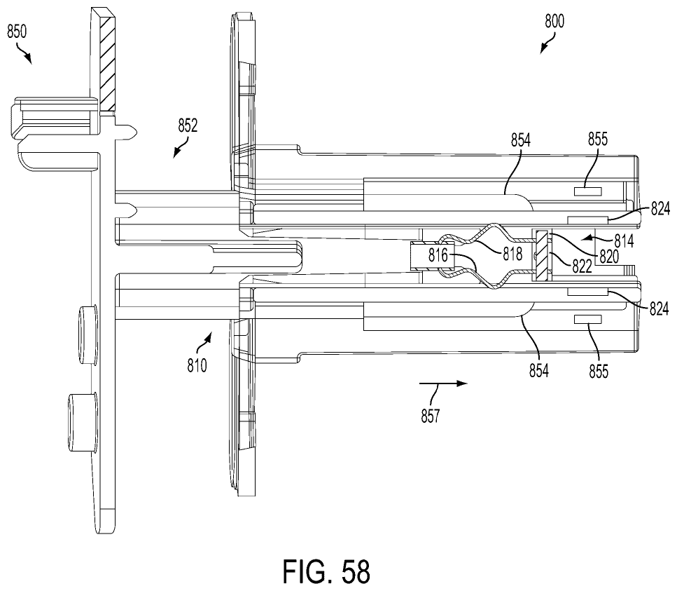

The contacts 816, 818 of the drain 814 may be coupled at a base portion 820 of the drain 814. According to various embodiments, the contacts 816, 818 may be electrically shorted to one another, or may be electrically connected to one another via a resistive element 822. FIG. 58 illustrates one embodiment of the battery pack 800 being installed to a surgical instrument 850. The surgical instrument 850 may comprise an extending member 852 configured to be received into the interior cavity 810. The extending member 854 may comprise one or more electrodes 854 positioned to contact electrodes 855 of the battery pack 800 when the member 854 is completely installed. In this way, the cells 812 of the battery pack 800 may provide electrical power to the instrument 830 via the electrodes 854, 855.

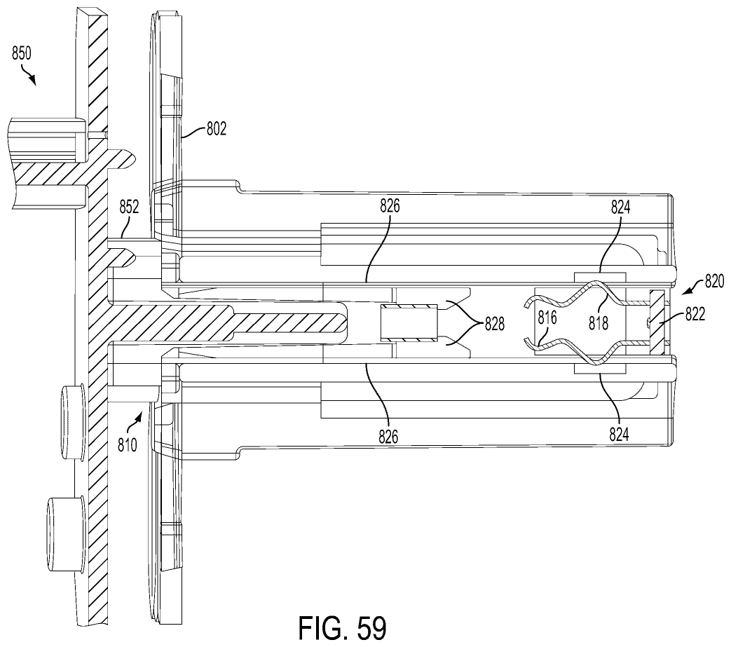

As the member 852 is inserted into the interior cavity 810, it may contact the battery drain 820 and force it along the interior cavity 810 in the direction of the arrow 857. For example, the force provided to the battery drain 820 by the member 852 may overcome the drain's resistance to movement provided by the contacts 816, 818, for example, in conjunction with the catch members 828. When completely installed, as shown in FIG. 59, the member 852 may push the drain 814 into the cavity 810 until the contacts 816, 818 come into electrical contact with the electrodes 824. This may either short the cells 812 or electrically connect them across the resistive element 822. When the battery pack 800 is uninstalled from the instrument 850, the member 852 may be removed from the cavity 810. The drain 814, however, may remain in the position shown in FIG. 59. In this way, the cells 812 may drain any remaining charge across the resistive element 822 either before or during disposal. This may, for example, minimize the power of any accidental discharges during disposal.

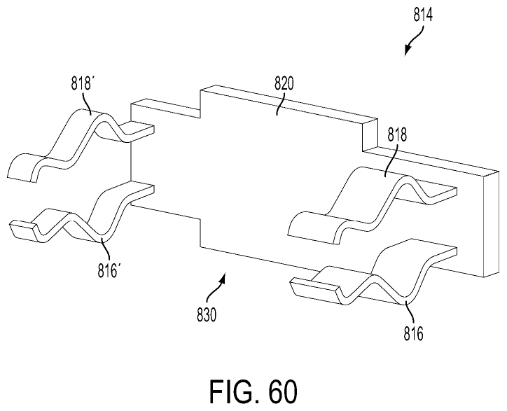

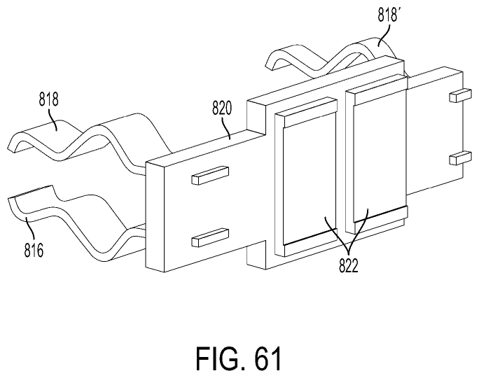

FIGS. 60 and 61 illustrates one embodiment of the battery drain 814 removed from the casing 802. As illustrated, the drain 814 may comprise two sets of contacts 818, 816 and 818′, 816′. The base 820 may define a central portion 830 between the two sets of contacts 816, 818, 816′, 818′. According to various embodiments, the central portion 830 may be configured to contact the member 852, as illustrated in FIGS. 58-59. Referring now to FIG. 61, resistive elements 822 are shown mounted to the base 820. The resistive elements 822 may be elements of any suitable resistance value and any suitable mechanical configuration. For example, as illustrated in FIG. 61, the resistive elements 822 may comprise one or more surface-mount components.

It is to be understood that at least some of the figures and descriptions herein have been simplified to illustrate elements that are relevant for a clear understanding of the disclosure, while eliminating, for purposes of clarity, other elements. Those of ordinary skill in the art will recognize, however, that these and other elements may be desirable. However, because such elements are well known in the art, and because they do not facilitate a better understanding of the disclosure, a discussion of such elements is not provided herein.

While several embodiments have been described, it should be apparent, however, that various modifications, alterations and adaptations to those embodiments may occur to persons skilled in the art with the attainment of some or all of the advantages of the disclosure. For example, according to various embodiments, a single component may be replaced by multiple components, and multiple components may be replaced by a single component, to perform a given function or functions. This application is therefore intended to cover all such modifications, alterations and adaptations without departing from the scope and spirit of the disclosure as defined by the appended claims.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A surgical instrument system, comprising:
a surgical instrument comprising an open electrical circuit; and
a battery pack, comprising:
a first battery cell; and
a second battery cell, wherein the assembly of said battery pack to said surgical instrument is configured to close said electrical circuit and electrically couple said first battery cell to said second battery cell.

2. The surgical instrument system of claim 1, wherein said surgical instrument comprises a handle, wherein said handle comprises a cavity, and wherein said battery pack is at least partially insertable into said cavity.

3. The surgical instrument system of claim 1, wherein said battery pack further comprises a third battery cell in electrical communication with said first battery cell prior to the assembly of said battery pack to said surgical instrument.

4. The surgical instrument system of claim 3, wherein said battery pack further comprises a fourth battery cell in electrical communication with said second battery cell prior to the assembly of said battery pack to said surgical instrument.

5. The surgical instrument system of claim 1, further comprising a drain configured to place said first battery cell in electrical communication with said second battery cell when said battery pack is assembled to said surgical instrument.

6. The surgical instrument system of claim 1, wherein said surgical instrument further comprises a shaft and an end effector extending from said shaft.

7. The surgical instrument system of claim 1, further comprising a staple cartridge.

8. A surgical instrument system, comprising:
a surgical instrument, comprising:
a first electrical contact; and
a second electrical contact; and
a battery pack, comprising:
a first independent battery cell; and
a second independent battery cell, wherein the assembly of said battery pack to said surgical instrument is configured to place said first independent battery cell in electrical communication with said first contact, said second independent battery cell in electrical communication with said second contact, and said first independent battery cell in electrical communication with said second independent battery cell.

9. The surgical instrument system of claim 8, wherein said surgical instrument comprises a handle, wherein said handle comprises a cavity, and wherein said battery pack is at least partially insertable into said cavity.

10. The surgical instrument system of claim 8, wherein said battery pack further comprises a third battery cell in electrical communication with said first independent battery cell prior to the assembly of said battery pack to said surgical instrument.

11. The surgical instrument system of claim 10, wherein said battery pack further comprises a fourth battery cell in electrical communication with said second independent battery cell prior to the assembly of said battery pack to said surgical instrument.

12. The surgical instrument system of claim 8, further comprising a drain configured to place said first independent battery cell in electrical communication with said second independent battery cell when said battery pack is assembled to said surgical instrument.

13. The surgical instrument system of claim 8, wherein said surgical instrument further comprises a shaft and an end effector extending from said shaft.

14. The surgical instrument system of claim 8, further comprising a staple cartridge.

15. A surgical instrument system, comprising:
a surgical instrument comprising an open electrical circuit; and
a battery pack, comprising:
a first battery; and
a second battery, wherein the assembly of said battery pack to electrically couple said first battery to said second battery.

16. The surgical instrument system of claim 15, wherein said surgical instrument comprises a handle, wherein said handle comprises a cavity, and wherein said battery pack is at least partially insertable into said cavity.

17. The surgical instrument system of claim 15, wherein said battery pack further comprises a third battery in electrical communication with said first battery prior to the assembly of said battery pack to said surgical instrument.

18. The surgical instrument system of claim 17, wherein said battery pack further comprises a fourth battery cell in electrical communication with said second battery cell to the assembly of said battery pack to said surgical instrument.

19. The surgical instrument system of claim 15, further comprising a drain configured to place said first battery in electrical communication with said second battery when said battery pack is assembled to said surgical instrument.

20. The surgical instrument system of claim 15, wherein said surgical instrument further comprises a shaft and an end effector extending from said shaft.
